(19) United States
(12) Patent Application Publication
(10) Pub. No.: US 2016/0243112 A1
(43) Pub. Date: Aug. 25, 2016

(54) TREATMENTS FOR ALZHEIMER'S RELATED DISEASES AND DISORDERS

(71) Applicant: Alkermes, Inc., Waltham, MA (US)

(72) Inventors: Daniel Deaver, Franklin, MA (US); David Eyerman, Wayland, MA (US); Thomas Andrew Wynn, Lexington, MA (US)

(21) Appl. No.: 15/052,118

(22) Filed: Feb. 24, 2016

Related U.S. Application Data
(60) Provisional application No. 62/120,710, filed on Feb. 25, 2015.

(51) Int. Cl. A61K 31/485; A61K 45/06; A61K 9/00

(52) U.S. Cl. CPC A61K 31/485; A61K 9/0053; A61K 45/06

(57) ABSTRACT

The invention relates to a method for the treatment of neuropsychiatric behavioral symptoms in patients with Alzheimer's Disease wherein the symptoms are selected from the group consisting of anxiety, agitation, aggression, depression, hallucination, memory loss, confusion, repetition, sleep issues, sun downing, suspicion, delusions, and wandering, comprising the step of administering to the patient an effective amount of a compound of Formula I, IA, IB, Table 1, preferably Compound-1, or a pharmaceutically acceptable salt thereof.

### RELATED APPLICATION

This application claims the benefit of U.S. Provisional Application No. 62/120,710, filed on Feb. 25, 2015. The entire teachings of the above application are incorporated herein by reference.

### BACKGROUND OF THE INVENTION

Alzheimer's disease (AD) is the most common form of dementia and is a progressive neurodegenerative disease eventually leading to death. An estimated 5.4 million Americans have AD, this number has doubled since 1980, and is expected to be as high as 16 million by 2050 (Hebert L. E. et al., (2001) Annual incidence of Alzheimer disease in the United States projected to the years 2000 through 2050. Alzheimer Dis Assoc Disord, 15(4):169-73). AD is generally characterized by cognitive decline, impaired performance of daily activities, and behavioral disturbances. In addition, frequent and severe neuropsychiatric behavioral symptoms, such as anxiety, agitation, aggression, depression, hallucination, memory loss, confusion, repetition, sleep issues, sun downing, suspicion, delusions, and wandering can be extremely distressing to the individual, the family, and caregivers.

Currently, a combination of dextromethorphan and quinidine has completed Phase 2 clinical studies for the treatment of Alzheimer's agitation. (AVP923: http://www.nia.nih.gov/alzheimers/clinical-trials/avp-923-treatment-agitation-alzheimers-patients; Alzheimer's Agitation Drug AVP-923 Clears Next Hurdle, Alzheimer's & Dementia Weekly, November 2014). AVP-923 is a fixed-dose combination of two approved drugs. The active ingredient is dextromethorphan hydrobromide (DM), an uncompetitive N-methyl-D-aspartate (NMDA) receptor antagonist and sigma-1 agonist and the enzyme inhibitor quinidine sulfate (Q), a CYP450 2D6 inhibitor, which serves to increase the bioavailability of dextromethorphan. The combination is currently commercialized under the name NUEDEXTA® for the treatment of pseudobulbar affect (PBA). NUEDEXTA® capsules contain either 20 mg or 30 mg of dextromethorphan hydrobromide with 10 mg of quinidine sulfate.

Dextromethorphan is also commercialized as an over the counter antitussive (cough suppressant). Since dextromethorphan is a substrate of the cytochrome P450 enzyme 2D6 and is rapidly metabolized, oral administration of dextromethorphan by itself leads to rapid and unsustained peak levels of drug with a short duration of action. (Stahl S. M., (2013) Mechanism of action of dextromethorphan/quinidine: comparison with ketamine CNS Spectrums, 225-227). Stahl also states that “oral administration of dextromethorphan alone does not lead to the sustained levels needed to substantially occupy neurotransmitter receptors in the brain without the need for frequent administration and high-peak doses associated with dissociative side effects.” When used recreationally, dextromethorphan acts as a dissociative hallucinogen when administered exceeding label-specified maximum dosages. At doses much higher than medically recommended, dextromethorphan and its major metabolite, dextrorphan, acts as an NMDA receptor antagonist, which produces effects similar to, yet distinct from, the dissociative hallucinogenic states created by other dissociative anesthetics such as ketamine and phencyclidine (PCP). As such, in the case of NUDEXTA®, an inhibitor of cytochrome P450 enzyme 2D6 such as quinidine sulfate is needed to slow down the metabolism of dextromethorphan and to “attain high degrees of continuous occupancy of brain receptors without frequent dosing and without peak dose dissociative side effects.” (Stahl, S. M., 2013).

Uncompetitive or high affinity NMDA receptor antagonists, like dizocilpine (MK-801) and phencyclidine (PCP) have been implicated in neuronal injury and excitotoxic cell death due in part to excessive activation of NMDA-type glutamate receptors and excessive Ca²⁺ influx through the receptor's associated ion channel (Lipton, S. A., (2004) Failures and Successes of NMDA Receptor Antagonists: Molecular Basis for the Use of Open-Channel Blockers like Memantine in the Treatment of Acute and Chronic Neurologic Insults NeuroRx, 1(1): 101-110). Uncompetitive NMDA receptor antagonists that block virtually all NMDA receptor activity will very likely have unacceptable clinical side effects. However, low-affinity, use-dependent NMDA receptor antagonists may have reduced toxicities because they reach a steady-state block more rapidly due to their rapid on-off kinetics (Rogawski, M. A., (1993) Therapeutic potential of excitatory amino acid antagonists: channel blockers and 2,3-benzodiazepines. Trends Pharmacol Sci, 14: 325-331), thus preventing significant calcium entry before equilibrium is reached without producing a supramaximal blockade. It has been suggested that the low-affinity, use-dependent NMDA antagonists, memantine (Muller W. E. et al., (1995) Noncompetitive NMDA receptor antagonists with fast open-channel blocking kinetics and strong voltage dependency as potential therapeutic agents for Alzheimer's dementia Pharmacopsychiatry, 28:113-124; Parsons C. G., et al., (1995) Comparison of the potency, kinetics and voltage-dependency of a series of uncompetitive NMDA receptor antagonists in vitro with anticonvulsive and motor impairment activity in vivo. Neuropharmacology, 34:1238-1258), amantadine (Parsons et al., 1995), and ADCI (Rogawski, M. A., et al., (1995) Anticonvulsant efficacy of ADCI (5-aminocarbonyl-10, 11-dihydro-5H-dibenzo[a,d]cyclohepten-5,10-imine) after acute and chronic dosing in mice. Epilepsia, 36:566-571), lack serious side effects due to their relatively rapid kinetics (Chen et al., (1992) Open-channel block of N-methyl-D-aspartate (NMDA) responses by memantine: Therapeutic advantage against NMDA receptor-mediated toxicity. J Neurosci, 12: 4427-4435). As such, there is a need to develop low trapping NMDA inhibitors that can alleviate one or more neuropsychiatric behavioral symptoms of Alzheimer's disease with improved therapeutic index and with minimal neurodegeneration.

### SUMMARY OF THE INVENTION

The invention relates to the alleviation of one or more neuropsychiatric behavioral symptoms associated with Alzheimer's Diseases. The invention relates to a method for the alleviation of neuropsychiatric behavioral symptoms in patients with Alzheimer's Disease with one or more behavioral disturbances wherein the behavioral symptoms are selected from the group consisting of anxiety, agitation, aggression, depression, hallucination, memory loss, confusion, repetition, sleep issues, sun downing, suspicion, delusions, and wandering, comprising the step of administering to the patient an effective amount of a compound of Formula I, or a compound of Table 1, preferably Compound-1, or a pharmaceutically acceptable salt, ester, solvate or derivative thereof.

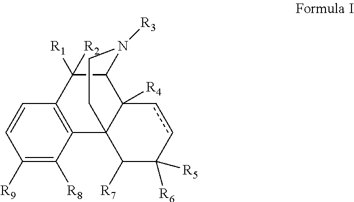

wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl; alternatively, R₅ and R₆ together form a carbonyl or a vinyl substituent;
each R₂₀ and R₂₁ is independently selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
Alternatively R₇ and R₈ taken together form an —O— or —S— group;
R₉ is selected from —C(O)N(R₂₀)(R₂₁), —C(S)N(R₂₀)(R₂₁), —C(O)NH₂, —C(O)N(R₂₀)N(R₂₀)C(O)R₂₁, aryl, substituted aryl, heterocyclyl and substituted heterocyclyl.

The invention relates to the treatment of N-methyl-D-aspartate (NMDA)-receptor related diseases or disorders by the administration of a compound described herein, particularly a compound of Formula I, IA, IB, Table 1, or Compound-1.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1: Chantest FLIPR Ca++Flux Assay in NR1/2a Expressing HEK Cells. The IC₅₀ of MK-801 (1.2 μM), DMX (106.8 μM), Ketamine (57.2 μM) and Compound-1 (225 μM) were determined from the assay.

FIG. 2: CEREP NMDA PCP Site binding study results showing specific binding comparison between dextromethorphan (DMX) and Compound-1.

FIG. 3: Effect of dextromethorphan on locomotor activity in the OBX rat. Drug treatment was given 30 minutes before test (1 mL/kg, s.c.). Data is expressed as Mean+SEM. #p<0.05 compared to vehicle treated control rats; * p<0.05 compared to vehicle-treated OBX rats.

FIG. 4: Effect of olfactory bulbectomy on locomotor activity in the open field. Drug treatment was given 30 minutes before test (1 mL/kg, s.c.).

FIG. 5: Effect of Compound-1 on locomotor activity in the OBX rat. Drug treatment was given 30 minutes before test (single s.c.administration). Drug treatment was given 30 minutes before test (single s.c.administration). Data expressed as Mean+SEM. *p<0.05; **p<0.01 compared to vehicle-treated OBX rats.

FIG. 6: Effect of olfactory bulbectomy on burying behavior. Drug treatment was given 30 minutes before test (1 mL/kg, s.c.). Data expressed as Mean+SEM.

FIG. 7: Effect of Compound-1 and dextromethorphan on burying behavior in OBX rat. Data expressed as Mean+SEM. **p<0.01 compared to vehicle-treated OBX rats.

FIG. 8: Effect of Compound-1 on locomotor activity in a Sprague Dawley rat. Drug treatment was given 30 minutes before test (1 mL/kg, s.c.).

FIG. 9: Effect of Compound-1 on jumping behavior in a Sprague Dawley rat. Drug treatment was given 30 minutes before test (single s.c.administration).

FIG. 10: Effect of Compound-1 on stereotypy in a Sprague Dawley rat. Drug treatment was given 30 minutes before test (single s.c.administration).

FIG. 11: Effect of Compound-1 on rearing in a Sprague Dawley rat. Drug treatment was given 30 minutes before test (single s.c.administration).

FIG. 12: Effect of Compound-1 on average velocity of a Sprague Dawley rat. Drug treatment was given 30 minutes before test (single s.c.administration).

Abbreviations: OBX=Olfactory bulbectomy rat; VEH=vehicle; DMX=Dextromethorphan; s.c.=subcutaneous.

### DETAILED DESCRIPTION OF THE INVENTION

In one embodiment, the invention relates to alleviating a neuropsychiatric behavioral symptom associated with Alzheimer's disease. The invention relates to a method for alleviating a neuropsychiatric behavioral symptom associated with Alzheimer's disease with one or more behavioral disturbances wherein the behavioral symptoms are selected from the group consisting of anxiety, agitation, aggression, depression, hallucination, memory loss, confusion, repetition, sleep issues, sun downing, suspicion, delusions, and wandering, comprising the step of administering to a subject in need thereof, an effective amount of a compound of Formula I, or a compound of Table 1, preferably Compound-1, or a pharmaceutically acceptable salt, ester, solvate or derivative thereof:

TABLE 1

| | |
|---|---|
| 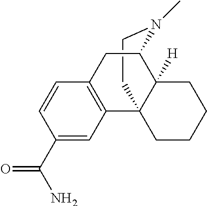 [SMILES: CN1CC[C@@]23CCCC[C@@H]2[C@@H]1Cc1ccc(C(N)=O)cc13] | Compound-1 |
| 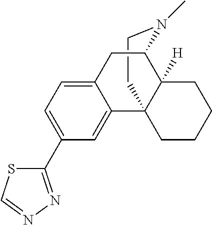 [SMILES: CN1CC[C@@]23CCCC[C@@H]2[C@@H]1Cc1ccc(-c2nncs2)cc13] | Compound-2 |
| 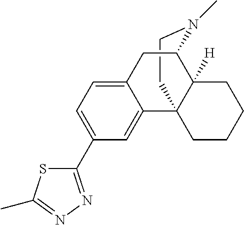 [SMILES: Cc1nnc(-c2ccc3c(c2)[C@]24CCCC[C@@H]2[C@H](C3)N(C)CC4)s1] | Compound-3 |

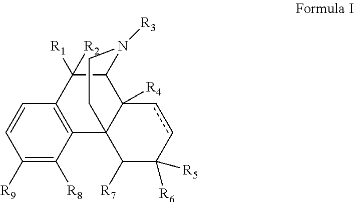

wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; preferably,
R₃ is selected from —CH₃, —(CH₂)ₙ-c-C₃H₅, —(CH₂)ₙ-c-C₄H₇, —(CH₂)ₙ-c-C₅H₉, —(CH₂)ₙ—CH═CH₂ and —(CH₂)ₙ—CH═C(CH₃)₂ wherein n is independently 0, 1, 2 or 3;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively R₅ and R₆ together form a carbonyl or a vinyl substituent;
each R₂₀ and R₂₁ is selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group;
R₉ is selected from —C(O)N(R₂₀)(R₂₁), —C(S)N(R₂₀)(R₂₁), —C(O)N(R₂₀)N(R₂₀)C(O)R₂₁, aryl, substituted aryl, heteroaryl, substituted heteroaryl, heterocyclyl and substituted heterocyclyl; preferably, —C(O)NH₂.

In one embodiment, the invention relates to the treatment of an NMDA-receptor related disease or disorder comprising the administration of a compound of Formula I, IA, IB, Table 1 or preferably, Compound-1:

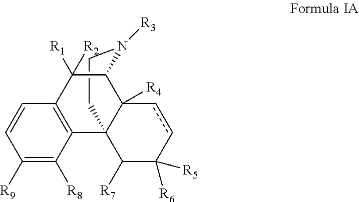

wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; Preferably,
R₃ is selected from —CH₃, —(CH₂)ₙ-c-C₃H₅, —(CH₂)ₙ-c-C₄H₇, —(CH₂)ₙ-c-C₅H₉, —(CH₂)ₙ—CH═CH₂ and —(CH₂)ₙ—CH═C(CH₃)₂ wherein n is independently 0, 1, 2 or 3;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl; alternatively R₅ and R₆ together form a carbonyl or a vinyl substituent;
each R₂₀ and R₂₁ is independently selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group;
R₉ is selected from —C(O)N(R₂₀)(R₂₁), —C(S)N(R₂₀)(R₂₁), —C(O)N(R₂₀)N(R₂₀)C(O)R₂₁, aryl, substituted aryl, heteroaryl, substituted heteroaryl, heterocyclyl and substituted heterocyclyl;

### Preferably, —C(O)NH₂.

In a further embodiment, the invention relates to the treatment of an NMDA-receptor related disease or disorder comprising the administration of a compound of Formula IA or a pharmaceutically acceptable salt thereof, wherein:

[symbol]

represents a single bond;
R₁ and R₂ are both hydrogen;
R₃ is selected from —CH₃, —(CH₂)ₙ-c-C₃H₅, —(CH₂)ₙ-c-C₄H₇, —(CH₂)ₙ-c-C₅H₉, —(CH₂)ₙ—CH═CH₂ and —(CH₂)ₙ—CH═C(CH₃)₂ wherein n is independently 0, 1, 2 or 3;
R₄ is hydrogen;
R₅ and R₆ are each hydrogen;
R₇ and R₈ are each hydrogen; and
R₉ is —C(O)NH₂.

In a further embodiment, the invention relates to the treatment of an NMDA-receptor related disease or disorder comprising the administration of a compound of Formula IA or a pharmaceutically acceptable salt thereof, wherein:

[symbol]

represents a single bond;
R₁ and R₂ are both hydrogen;
R₃ is hydrogen or alkyl;
R₄ is hydrogen;
R₅ and R₆ are each hydrogen;
R₇ and R₈ are each hydrogen; and
R₉ is —C(O)NH₂.

In a further embodiment, the invention relates to the treatment of an NMDA-receptor related disease or disorder comprising the administration of a compound of Formula IA or a pharmaceutically acceptable salt thereof, wherein:

[symbol]

represents a single bond;
R₁ and R₂ are both hydrogen;
R₃ is alkyl;
R₄ is hydrogen;
R₅ and R₆ are each hydrogen;
R₇ and R₈ are each hydrogen; and
R₉ is —C(O)NH₂.

In a further embodiment, the invention relates to the treatment of an NMDA-receptor related disease or disorder comprising the administration of a Compound of Formula IB or a pharmaceutically acceptable salt thereof:

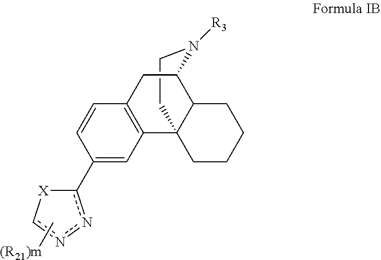

[symbol]

wherein, represents a single or double bond;
m is 0, 1, 2 or 3;
X is CH₂, NR₂₀, S, S(O)₂ or O; preferably, S;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; preferably, —CH₃, —(CH₂)ₙ-c-C₃H₅, —(CH₂)ₙ-c-C₄H₇, —(CH₂)ₙ-c-C₅H₉, —(CH₂)ₙ—CH═CH₂ or —(CH₂)ₙ—CH═C(CH₃)₂ wherein n is independently 0, 1, 2 or 3;
R₂₁ is selected from hydrogen, halogen, aliphatic, substituted aliphatic, aryl and substituted aryl.

In a further embodiment, the invention relates to the treatment of an NMDA-receptor related disease or disorder comprising the administration of a compound of Formula IB or a pharmaceutically acceptable salt thereof, wherein:

[symbol]

represents a single bond;
m is 0;

### X is S; and

R₃ is hydrogen or alkyl.

In a further embodiment, the invention relates to the treatment of an NMDA-receptor related disease or disorder comprising the administration of a compound of Formula IB or a pharmaceutically acceptable salt thereof, wherein:

[symbol]

represents a single bond;
m is 0;
X is S; and,
R₃ is alkyl.
In one embodiment, the compound of Formula I is selected from:

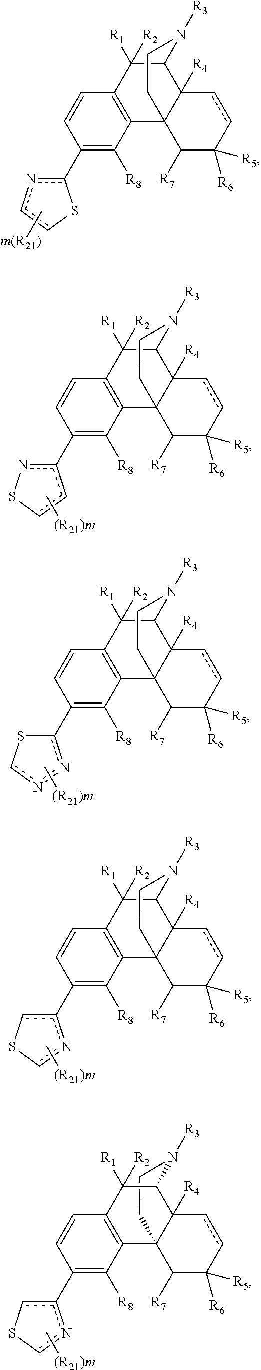

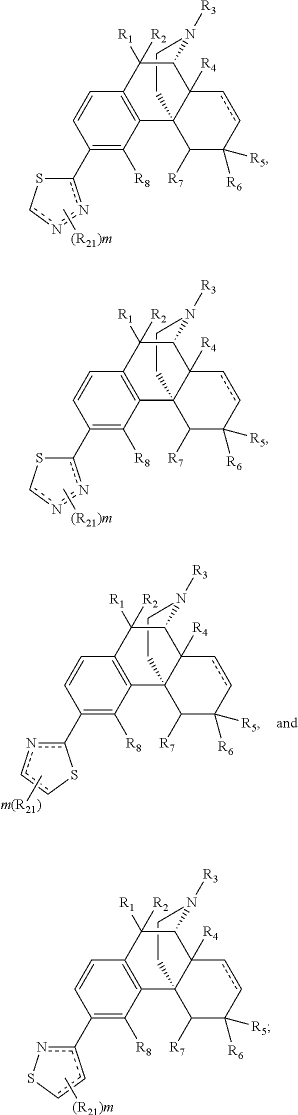

wherein, [symbol] represents a single or double bond;
m is 0, 1, 2 or 3;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; preferably,
R₃ is selected from —CH₃, —(CH₂)ₙ-c-C₃H₅, —(CH₂)ₙ-c-C₄H₇, —(CH₂)ₙ-c-C₅H₉, —(CH₂)ₙ—CH═CH₂ and —(CH₂)ₙ—CH═C(CH₃)₂ wherein n is independently 0, 1, 2 or 3;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₅ and R₆ together form a carbonyl or a vinyl substituent;
each R₂₀ and R₂₁ is independently selected from hydrogen, halogen, aliphatic, substituted aliphatic, aryl or substituted aryl; alternatively, two R₂₀ groups or one R₂₀ and one R₂₁ groups together with the atoms to which they are attached may form a two, three, four, five, six or seven membered ring; and
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl; alternatively, R₇ and R₈ together form an —O— or —S— group.

In another embodiment, the invention relates to a method of alleviating a neuropsychiatric behavioral symptom associated with Alzheimer's disease comprising administering to a patient in need thereof an effective amount of a compound of Formula I, or a compound of Table 1, preferably Compound-1, or a pharmaceutically acceptable salt, ester, solvate or derivative thereof.

In another embodiment, the invention relates to a method of alleviating a neuropsychiatric behavioral symptom associated with Alzheimer's disease comprising administering to a patient in need thereof an effective amount of a compound of Formula I or a pharmaceutically acceptable salt thereof.

In another embodiment, the invention relates to a method of alleviating a neuropsychiatric behavioral symptom associated with Alzheimer's disease comprising administering to a patient in need thereof an effective amount of a compound of Formula IA or a pharmaceutically acceptable salt thereof.

In another embodiment, the invention relates to a method of alleviating a neuropsychiatric behavioral symptom associated with Alzheimer's disease comprising administering to a patient in need thereof an effective amount of a compound of Formula IB or a pharmaceutically acceptable salt thereof.

In another embodiment, the invention relates to a method of alleviating a neuropsychiatric behavioral symptom associated with Alzheimer's disease comprising administering to a patient in need thereof an effective amount of compound-1, or a pharmaceutically acceptable salt thereof.

In another embodiment, the invention relates to a compound of Formula I, or a compound of Table 1, preferably compound-1, or a pharmaceutically acceptable salt, ester, solvate or derivative thereof for use in treatment of a neuropsychiatric behavioral symptom associated with Alzheimer's disease.

In another embodiment, the invention relates to a compound of Formula I or a pharmaceutically acceptable salt thereof for use in treatment of a neuropsychiatric behavioral symptom associated with Alzheimer's disease.

In another embodiment, the invention relates to a compound of Formula IA or a pharmaceutically acceptable salt thereof for use in treatment of a neuropsychiatric behavioral symptom associated with Alzheimer's disease.

In another embodiment, the invention relates to a compound of Formula IB or a pharmaceutically acceptable salt thereof for use in treatment of a neuropsychiatric behavioral symptom associated with Alzheimer's disease.

In another embodiment, the invention relates to compound-1 or a pharmaceutically acceptable salt thereof for use in treatment of a neuropsychiatric behavioral symptom associated with Alzheimer's disease.
In another embodiment, the invention relates to a compound of Formula IB or a pharmaceutically acceptable salt thereof:

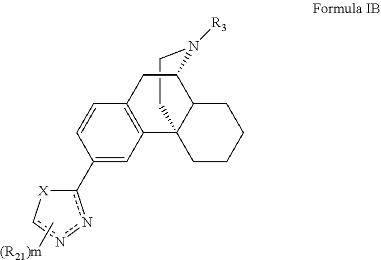

wherein, [symbol] represents a single or double bond;
m is 0, 1, 2 or 3;
X is CH₂, NR₂₀, S, S(O)₂ or O; preferably, S;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; preferably, —CH₃, —(CH₂)ₙ-c-C₃H₅, —(CH₂)ₙ-c-C₄H₇, —(CH₂)ₙ-c-C₅H₉, —(CH₂)ₙ—CH═CH₂ or —(CH₂)ₙ—CH═C(CH₃)₂ wherein n is independently 0, 1, 2 or 3; and,
R₂₁ is selected from hydrogen, halogen, aliphatic, substituted aliphatic, aryl and substituted aryl.

In a further embodiment, the invention relates to a compound of Formula IB or a pharmaceutically acceptable salt thereof, wherein:

[symbol]

represents a single bond;
m is 0;
X is S; and,
R₃ is hydrogen or alkyl.

In a further embodiment, the invention relates to a compound of Formula IB or a pharmaceutically acceptable salt thereof, wherein:

[symbol]

represents a single bond;
m is 0;
X is S; and,
R₃ is alkyl.

Applicants have surprisingly discovered that administration of an effective amount of a compound of Formula I, or a compound of Table 1, preferably Compound-1, a low trapping N-methyl-D-aspartate (NMDA) channel blocker, is effective at alleviating a neuropsychiatric behavioral symptom associated with Alzheimer's disease. As described herein, the term “low trapping N-methyl-D-aspartate (NMDA) channel blocker” is a compound that has low to moderate affinity to the PCP site of the NMDA channel as exhibited by a binding affinity (Ki) range of 1-100 μM and IC₅₀ range of 30-300 μM in an FLIPR® Calcium Flux Assay. The FLIPR® Calcium Flux Assay provides a method of assessing glutamate NMDA receptor activity through changes in intracellular calcium (Ca²⁺) concentration. The CEREP PCP antagonist radioligand assay assesses the ability to inhibit specific binding of phencyclidine to the PCP binding site of NMDA receptors in rat cerebral cortex cells. Due to the high site to site variability used in assays to characterize NMDA antagonists, the comparisons of compounds need to be made within the same assay system. In a preferred embodiment, the binding affinity (Ki) range is 5-50 μM. In a more preferred embodiment, the binding affinity (Ki) range is 10-25 μM. In another preferred embodiment, the IC₅₀ range is 100-300 μM. In yet a more preferred embodiment, the IC₅₀ range is 150-300 μM.

As used herein, the terms “compound”, “drug”, and “prodrug” all include pharmaceutically acceptable salts, co-crystals, solvates, hydrates, polymorphs, enantiomers, diastereoisomers, racemates and the like of the compounds, drugs and prodrugs having the formulas as set forth herein.

Alzheimer's disease (AD) is a type of dementia that causes problems with memory, thinking and behavior. Symptoms usually develop slowly and get worse over time, becoming severe enough to interfere with daily tasks. As described herein, the term “neuropsychiatric behavioral symptom associated with Alzheimer's disease” includes anxiety, agitation, aggression, depression, hallucination, memory loss, confusion, repetition, sleep issues, sun downing, suspicion, delusions, and wandering. In particular, a person with AD may feel anxious or agitated. He or she may become restless, causing a need to move around or pace, or become upset in certain places or when focused on specific details. A person with AD may also exhibit aggressive behavior, which may be verbal (e.g., screaming, cussing) or physical (e.g., destroying objects, grabbing, fighting). They can occur suddenly, with no apparent reason, or result from a frustrating situation. Hallucinations are false perceptions of objects or events involving the senses. When a person with AD hallucinates, he or she may see, hear, smell, taste or feel something that isn't there. A person with AD may also do or say something over and over, like repeating a word, question or activity, or undo something that has just been finished. People with AD may have problems sleeping or have increases in behavioral problems that begin at dusk and last into the night (known as sundowning). In addition, a person with AD may suffer delusions (firmly held beliefs in things that are not real) or become suspicious of those around them, even accusing others of theft, infidelity or other improper behavior. Confusion and memory loss, such as the inability to remember certain people or objects, can contribute to these untrue beliefs. Lastly, a person with AD may wander. They may not remember his or her name or address, and can become disoriented, even in familiar places.

As used herein, the term “alleviate,” when used in reference to an undesirable or adverse symptom or complication (physiological or psychological), means a detectable or measurable therapeutic benefit to a subject. A therapeutic benefit is any objective or subjective transient or temporary, or longer term improvement in the subject's physiological or psychological condition. For example, a satisfactory clinical endpoint is achieved when there is an incremental improvement in the subject's condition, or a reduction or stabilization (inhibiting a progression or worsening of the condition) of the frequency, severity or duration of one or more undesirable or adverse symptoms (i.e., undesirable or adverse symptoms) or a physiological or psychological complication associated with or caused by the condition, or a stabilization, inhibition or reversal of one or more of the physiological, biochemical or cellular manifestations or characteristics associated with or caused by the condition.

To “alleviate” one or more neuropsychiatric behavioral symptoms associated with Alzheimer's disease therefore includes any reduction, inhibition, stabilization or prevention of an undesirable or adverse behavioral symptom selected from the group consisting of anxiety, agitation, aggression, depression, hallucination, memory loss, confusion, repetition, sleep issues, sun downing, suspicion, delusions, and wandering. As such, the term “alleviate” does not require complete ablation of an undesirable or adverse behavioral symptom associated with Alzheimer's disease. For example, in the case of aggressive verbal or physical behavior, inhibiting or reducing the severity or incidence (frequency) of screaming, cussing, destroying objects, grabbing or fighting episodes provides a therapeutic benefit (e.g., less frequent bouts or a reduction from moderate to mild aggressive episodes) even though complete ablation of aggression may not result. An improvement in a subjects' subjective feeling, such as increased confidence, reduced depression, increased participation in outdoor or physical activities, and improved psychological wellbeing, are also examples of a therapeutic benefit.

In a more preferred embodiment the behavioral symptom being alleviated is anxiety. In another preferred embodiment the behavioral symptom being alleviated is agitation. In yet another preferred embodiment the behavioral symptom being alleviated is aggression.

The patient, as used herein is preferably a mammal, with human patients especially preferred, is suffering from one or more neuropsychiatric behavioral symptom associated with Alzheimer's disease.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

NMDA antagonists are associated with a wide range of serious toxicities, including neurodegeneration. However, the therapeutic index of an NMDA antagonist can vary widely and is correlated with a property referred to as trapping at the drug binding site within the NMDA receptor channel. Higher trapping agents show increased toxicity relative to lower trapping agents and a reduced therapeutic index. Compounds of Formula I, or a Compound of Table 1, preferably Compound-1, have been evaluated in repeat dose general toxicology studies of up to six weeks in duration in both rats and dogs. In both species, Compounds of Formula I, or a Compound of Table 1, preferably Compound-1, were given daily by oral gavage at doses producing plasma concentrations of the drug that would exceed the expected human therapeutic exposures by at least 30-fold. At the highest doses evaluated, Compounds of Formula I, or a Compound of Table 1, preferably Compound-1 produced clinical changes in rats (increased bone fractures and abnormalities in incisors) common to other NMDA antagonists. However, even when these clinical signs were present in rats, there was no evidence of neurodegeneration. This observation is consistent with the concept of the low trapping nature of Compounds of Formula I, or a Compound of Table 1, preferably Compound-1, and an improved therapeutic index over other NMDA antagonists characterized as high trapping agents.

NMDA antagonists are also implicated in neurodegenerative diseases and other neuro-related conditions as they cause neuronal loss and cognitive impairment. In particular, NMDA antagonists play a part in the final common pathway leading to neuronal injury in a variety of neurodegenerative disorders such as amyotrophic lateral sclerosis (ALS), Parkinson's disease, Alzheimer's disease (AD) and Huntington's disease, as well as conditions such as stroke, ischemia, CNS trauma, hypoxia, and hypoglycemia. Recent findings have further implicated NMDA receptors in many other neurological disorders, such as multiple sclerosis (MS), epilepsy (Rejdak, K. et al., (2014) Orphenadrine-induced convulsive status epilepticus in rats responds to the NMDA antagonist dizocilpine, Pharmacol Rep, 66(3):399-403), cerebral palsy (periventricular leukomalacia), and spinal cord injury, as well as in chronic, severe mood disorders, acute schizophrenia and treatment resistant schizophrenia, treatment resistant depression, bipolar depression, major depressive disorder (Hashimoto K. et al., (2013) Glutamate modulators as potential therapeutic drugs in schizophrenia and affective disorders, Eur. Arch Psychiatry Clin Neurosci, 263 (5): 367-377; Dang Y. H., (2014) Targeting of NMDA receptors in the treatment of major depression, Curr Pharm Des, 20(32):5151-9), obsessive compulsive disorders (OCD), drug addiction and other addictive behaviors (i.e., pathological gambling), anxiety disorders, autism spectrum disorders (Siegel et al., (2012) Pharmacology, Biochemistry and Behavior, 100(4): 653-655), pain management and suicidal ideation.

NMDA receptor mediated diseases or disorders include, but are not limited to, pseudobulbar affect (PBA), Alzheimer's disease, amyotrophic lateral sclerosis (ALS), Parkinson's disease, Huntington's disease, stroke, ischemia, CNS trauma, hypoxia, hypoglycemia, multiple sclerosis (MS), epilepsy, cerebral palsy (periventricular leukomalacia), spinal cord injury, chronic and severe mood disorders, acute and treatment resistant schizophrenia, bipolar depression, treatment resistant depression, major depressive disorder, obsessive compulsive disorder (OCD), drug addiction and other addictive behaviors, anxiety disorder, autism spectrum disorders, adult autism, pain management and suicidal ideation.

In another preferred embodiment, the invention relates to a method of alleviating a neuropsychiatric behavioral symptom associated with Alzheimer's disease, comprising administering a low trapping N-methyl-D-aspartate (NMDA) channel blocker to a subject in need thereof, wherein the binding affinity (Ki) range is between about 1-100 μM to the PCP binding site of the NMDA receptor, and the IC₅₀ range is between about 30 to 300 μM in a FLIPR calcium flux assay, wherein the method does not cause psychotomimetic liability or dissociative side effects. In a preferred embodiment, the IC₅₀ range is between about 100 to 300 μM, more preferably between about 150 to 300 μM. In a preferred embodiment, the binding affinity (Ki) range is between about 5-50 μM, more preferably between about 10-25 μM. In one embodiment, the psychotomimetic liability is visual hallucinations. In one embodiment, the dissociative side effect comprises feeling abnormal, disinhibition, illusion and dissociation.

In yet another preferred embodiment, the invention relates to a method of treating a disease mediated by an NMDA receptor, comprising administering a low trapping N-methyl-D-aspartate (NMDA) channel blocker to a subject in need thereof, wherein the binding affinity (Ki) range is between about 1-100 μM to the PCP binding site of the NMDA receptor, and the IC₅₀ range is 30 to 300 μM in a FLIPR calcium flux assay, wherein the method provides an improved therapeutic window with lower neural toxicity. In a preferred embodiment, the IC₅₀ range is between about 100 to 300 μM, more preferably between about 150 to 300 μM. In a preferred embodiment, the binding affinity (Ki) range is between about 5-50 μM, more preferably between about 10-25 μM.

In another embodiment, the invention relates to a method of treating a disease or disorder, or a symptom of a disease or disorder mediated by an N-methyl-D-aspartate (NMDA) receptor comprising the step of administering a compound of Formula I, or a pharmaceutically acceptable salt thereof, to a subject in need thereof.

In another embodiment, the invention relates to a method of treating a disease or disorder, or a symptom of a disease or disorder mediated by an N-methyl-D-aspartate (NMDA) receptor comprising the step of administering a compound of Formula IA, or a pharmaceutically acceptable salt thereof, to a subject in need thereof.

In another embodiment, the invention relates to a method of treating a disease or disorder, or a symptom of a disease or disorder mediated by an N-methyl-D-aspartate (NMDA) receptor comprising the step of administering a compound of Formula IB, or a pharmaceutically acceptable salt thereof, to a subject in need thereof.

In another embodiment, the invention relates to a method of treating a disease or disorder, or a symptom of a disease or disorder mediated by an N-methyl-D-aspartate (NMDA) receptor comprising the step of administering a compound selected from Table 1, or a pharmaceutically acceptable salt thereof, to a subject in need thereof.

In another embodiment, the invention relates to a method of treating a disease or disorder, or a symptom of a disease or disorder mediated by an N-methyl-D-aspartate (NMDA) receptor comprising the step of administering Compound-1, or a pharmaceutically acceptable salt thereof, to a subject in need thereof.

In another embodiment, the disease or disorder mediated by an N-methyl-D-aspartate (NMDA) receptor is Alzheimer's disease.

In another embodiment, the disease or disorder mediated by an N-methyl-D-aspartate (NMDA) receptor is pseudobulbar affect (PBA).

In another embodiment, the symptom of a disease or disorder mediated by an N-methyl-D-aspartate (NMDA) receptor is agitation or anxiety.

In another embodiment, the symptom of a disease or disorder mediated by an N-methyl-D-aspartate (NMDA) receptor is agitation.

In another embodiment, the symptom of a disease or disorder mediated by an N-methyl-D-aspartate (NMDA) receptor is agitation associated with Alzheimer's disease.

In another embodiment, the symptom of a disease or disorder mediated by an N-methyl-D-aspartate (NMDA) receptor is anxiety associated with Alzheimer's disease.

In another embodiment, the invention relates to a compound of Formula I, or a pharmaceutically acceptable salt thereof, for use in treatment of a disease or disorder, or a symptom of a disease or disorder mediated by an N-methyl-D-aspartate (NMDA) receptor.

In another embodiment, the invention relates to a compound of Formula IA, or a pharmaceutically acceptable salt thereof, for use in treatment of a disease or disorder, or a symptom of a disease or disorder mediated by an N-methyl-D-aspartate (NMDA) receptor.

In another embodiment, the invention relates to a compound of Formula IB, or a pharmaceutically acceptable salt thereof, for use in treatment of a disease or disorder, or a symptom of a disease or disorder mediated by an N-methyl-D-aspartate (NMDA) receptor.

In another embodiment, the invention relates to a compound selected from Table 1, or a pharmaceutically acceptable salt thereof, for use in treatment of a disease or disorder, or a symptom of a disease or disorder mediated by an N-methyl-D-aspartate (NMDA) receptor.

In another embodiment, the invention relates to compound-1, or a pharmaceutically acceptable salt thereof, for use in treatment of a disease or disorder, or a symptom of a disease or disorder mediated by an N-methyl-D-aspartate (NMDA) receptor.

As used herein, the term “effective amount of the subject compound,” with respect to the subject method of treatment, refers to an amount of the subject compound which, when delivered as part of desired dose regimen, brings about management of the disease or disorder to clinically acceptable standards.

In one embodiment, the invention alleviates a neuropsychiatric behavioral symptom associated with Alzheimer's disease. The amount of a compound of Formula I, IA, IB, or a compound of Table 1, preferably Compound-1, adequate to accomplish this is defined as a “therapeutically effective dose”. The dosage schedule and amounts effective for this use, i.e., the “dosing regimen,” will depend upon a variety of factors, including the patient's physical status, age and the like. In calculating the dosage regimen for a patient, the mode of administration also is taken into consideration. In a preferred embodiment, a Compound of Formula I, or a Compound of Table 1, preferably Compound-1, is administered in a daily dose of about 1 mg to about 300 mg/day. In another preferred embodiment, a Compound of Formula I, or a Compound of Table 1, preferably Compound-1 is administered orally.

In one embodiment, a compound of Formula I, IA, IB, a compound of Table 1 or preferably, Compound-1 is administered to a patient in conjunction with a second pharmaceutical agent, preferably selected from an acetyl choline esterase inhibitor (AChEI), in a non-anticholinergic and an antiemetic agent. The AChEI can be selected from the group consisting of tacrine, donepezil, rivastigmine, galantamine, and pharmaceutically acceptable salts thereof. In one embodiment, the second pharmaceutical agent is selected from acamprosate, amlodipine, argatroban, baclofen, cilostazol, cinacalcet, clopidogrel, dyphylline, fenoldopam, leflunomide, mepacrine, methimazole, phenformin, prilocaine, rifabutin, sulfisoxazole, tadalafil, terbinafine, torasemide, cinnarizine, ciclopirox, eplerenone, carbenoxolone, sulodexide, carbamazine, amobarbital, cefotetan, erythrityl tetranitrate, methyclothiazide, risedronate, enprofylline, oxtriphylline, paramethadione, cefmenoxime, aprindine, etomidate, mitiglinide, benidipine, levosimendan, zonisamide, and pharmaceutically acceptable salts thereof.

In one embodiment, a compound of Formula I, IA, IB, Table 1 or Compound-1 is administered to a patient in combination with Aricept, donepezil, Exelon, Razadyne, Exelon, galantamine, Razadyne, Aricept or Rivastigmine. In one embodiment, the invention relates to a composition comprising a compound of Formula I, IA, IB, Table 1 or Compound-1 and a compound selected from ARICEPT®, donepezil, EXELON®, RAZADYNE®, galantamine, or rivastigmine. In one embodiment, a compound of Formula I, IA, IB, Table 1 or Compound-1 is administered to a patient in combination with one or more of the cholinesterase inhibitors (ARICEPT®, EXELON®, RAZADYNE®, COGNEX®) and/or memantine (NAMENDA®) to treat the cognitive symptoms (memory loss, confusion, and problems with thinking and reasoning) of Alzheimer's disease.

Suitable daily oral dosages of a compound of Formula I, or a compound of Table 1, preferably Compound-1 are described herein and are on the order of about 1.0 mg to about 300 mg, or about 10 mg to about 100 mg. For example, about 10, 20, 30, 40, 50, 60, 70, 80, 90 or 100 mg of Compound-1 is dosed orally per day. Desirably, each oral dosage contains from 1.0 to 300 mgs, particularly 1.0, 2.5, 5, 10, 15, 20, 25, 30, 40, 50, 60, 70, 80, 90, 100, 110, 120, 130, 140, 150, 160, 170, 180, 190, 200, 210, 220, 230, 240, 250, 260, 270, 280, 290 or 300 milligrams of a Compound of Formula I, or a Compound of Table 1, preferably Compound-1 and are administered for alleviation of a neuropsychiatric behavioral symptoms associated with Alzheimer's disease. Dosage regimen may be adjusted to provide the optimal therapeutic response. The specific dose level for any particular patient will vary depending upon a variety of factors, including but not limited to, the activity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

Compounds of Formula I, IA, IB and Table 1, preferably Compound-1 can be administered in such oral forms as tablets, capsules (each of which includes sustained release or timed release formulations), pills, powders, granules, elixirs, tinctures, suspensions, syrups, and emulsions. The present invention includes the use of both oral rapid-release and time-controlled release pharmaceutical formulations (see, e.g., U.S. Pat. Nos. 6,495,166; 5,650,173; 5,654,008 which describes controlled release formulations and is incorporated herein by reference).

Compounds of Formula I, IA, IB and Table 1, preferably Compound-1 can be administered in a mixture with pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices. For instance, for oral administration in the form of a tablet or capsule, compound-1 can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents, coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like. For a discussion of dosing forms, carriers, additives, pharmacodynamics, etc., see Kirk-Othmer Encyclopedia of Chemical Technology, Fourth Edition, 1996, 18:480-590, incorporated herein by reference.

As used herein, the term “pharmaceutically acceptable carrier or excipient” means a non-toxic, inert solid, semi-solid, gel or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta-(β) and gamma-(γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir. In a preferred embodiment, administration is oral administration.

Liquid dosage forms for oral administration include pharmaceutically acceptable emulsions, microemulsions, solutions, suspensions, syrups and elixirs. In addition to the active compound, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1, 3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In another embodiment, administration is parenteral administration by injection. Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1, 3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing a compound of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. They may optionally contain embedding agents, preferably polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics is known in the art (see, for example U.S. Pat. No. 5,767,068 to VanDevanter et al., U.S. Pat. No. 5,508,269 to Smith et al., and WO 98/43650 by Montgomery).

### DEFINITIONS

The term “aliphatic group” or “aliphatic” refers to a non-aromatic moiety that may be saturated (e.g. single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted saturated aliphatic hydrocarbon radicals/groups having the specified number of carbons. Preferred alkyl groups comprise about 1 to about 24 carbon atoms (“C₁-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The “-c-” notation is used to designate cyclic groups. For example, —CH₂-c-C₃H₅ represents —CH₂-cyclopropyl.

The term “alkenyl” refers to linear or branched radicals having at least one carbon-carbon double bond. Such radicals preferably contain from about two to about twenty-four carbon atoms (“C₂-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means an aromatic system containing one, two or three rings wherein such rings may be attached together in a pendent manner or may be fused. The term “aryl” embraces aromatic radicals such as phenyl, naphthyl, tetrahydronaphthyl, indane furanyl, quinazolinyl, pyridyl and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1, 2, 4-triazolyl, 1H-1, 2, 3-triazolyl, 2H-1, 2, 3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1, 5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1, 2, 4-oxadiazolyl, 1, 3, 4-oxadiazolyl, 1, 2, 5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1, 2, 4-thiadiazolyl, 1, 3, 4-thiadiazolyl, 1, 2, 5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl’ refers to heterocyclo-substituted alkyl radicals. More preferred heterocycloalkyl radicals are “lower heterocycloalkyl’ radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are ‘lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms ‘aralkyl” or “arylalkyl’ refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term ‘alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are ‘lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N, N-alkylamino, such as N-methylamino, N-ethylamino, N, N-dimethylamino, N, N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkyl sulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl”, in which case those skilled in the art will understand the alkyl to be a divalent radical (e.g., —CH₂—CH₂—), which is equivalent to the term “alkylene.’ Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

The terms “compound” “drug,” and “prodrug” as used herein all include pharmaceutically acceptable salts, co-crystals, solvates, hydrates, polymorphs, enantiomers, diastereoisomers, racemates and the like of the compounds, drugs and prodrugs having the formulas as set forth herein.

The present invention includes all pharmaceutically acceptable isotopically-labeled or enriched compounds of the invention. The compounds include one or more atoms that are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, nitrogen, such as ¹³N and ¹⁵N, oxygen, such as ¹⁵O, ¹⁷O and ¹⁸O, chlorine, such as ³⁶Cl, fluorine, such as ¹⁸F, iodine, ¹²³I and ¹²⁵I, phosphorus, such as ³²P, and sulphur, such as ³⁵S.

Substituents indicated as attached through variable points of attachments can be attached to any available position on the ring structure.

These definitions provided in the application apply to the terms as they are used throughout this specification and claims, unless otherwise limited in specific instances, either individually or as part of a larger group.

### EXEMPLIFICATION

Compound-1 can be synthesized utilizing a process similar to those described in U.S. Pat. No. 6,784,187.

### Synthesis of Compound-2

### Synthesis of Compound-2a: Synthesis of (4bS,9S)-11-methyl-6,7,8,8a,9,10-hexahydro-5H-9,4b-(epiminoethano)phenanthren-3-ol

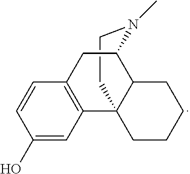 [SMILES: CN1CC[C@@]23CCCCC2[C@@H]1Cc1ccc(O)cc13]

A mixture of Dextromethorphan HBr salt (11 g, 29.7 mmol) in 48% HBr_{(aq)} (110 mL) was heated at 130° C. overnight. The reaction was allowed to cool to room temperature then quenched with a mixture of 28% ammonium hydroxide (approximately 500 mL) and sufficient ice to keep the temperature below 0° C. at all times. The product was extracted into dichloromethane (1 L) and dried (MgSO₄). Filtration and removal of the solvent under reduced pressure gave (4bS,9S)-11-methyl-6,7,8,8a,9,10-hexahydro-5H-9,4b-(epiminoethano)phenanthren-3-ol (7.48 g, 98% yield); ¹H NMR (300 MHz, CDCl₃) 6.95 (1H, d), 6.70 (1H, d), 6.60 (1H, dd), 2.96 (1H, d), 2.89-2.83 (1H, m), 2.63 (1H, dd), 2.50 (1H, dd), 2.41 (3H, s), 2.30-2.23 (1H, m), 2.16 (1H, td), 1.88 (1H, dt), 1.76 (1H, td), 1.66-1.57 (1H, m), 1.51-1.44 (1H, m), 1.43-1.24 (5H, m), 1.20-1.06 (1H, m).

### Synthesis of Compound-2b: Synthesis of (4bS,9S)-11-methyl-6,7,8,8a,9,10-hexahydro-5H-9,4b-(epiminoethano)phenanthren-3-yl trifluoromethanesulfonate

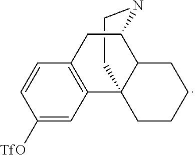 [SMILES: O=S(=O)(Oc1ccc2c(c1)[C@]13CCCCC1[C@H](C2)NCC3)C(F)(F)F]

A mixture of (4bS, 9S)-11-methyl-6,7,8,8a,9,10-hexahydro-5H-9,4b-(epiminoethano)phenanthren-3-ol (7.48 g, 29.1 mmol), N-phenylbis(trifluoromethanesulfonamide) (10.92 g, 30.6 mmol), and triethylamine (12.2 mL, 87.3 mmol) in dichloromethane (200 mL) was stirred at room temperature overnight. The mixture was concentrated under reduced pressure and the residue partitioned between ethyl acetate/heptane (5:1) (500 mL) and washed with water (5×250 mL) until none of the triflating reagent remained. The organic phase was dried (MgSO₄). Filtration and removal of the solvent under reduced pressure gave (4bS,9S)-11-methyl-6,7,8,8a,9,10-hexahydro-5H-9,4b-(epiminoethano)phenanthren-3-yl trifluoromethanesulfonate (11.32 g, 100% yield); [M+H]⁺390.42.

### Synthesis of Compound-2c: Synthesis of (4bS,9S)-11-methyl-3-(4,4,5,5-tetramethyl-1,3,2-dioxaborolan-2-yl)-6,7,8,8a,9,10-hexahydro-5H-9,4b-(epiminoethano)phenanthrene

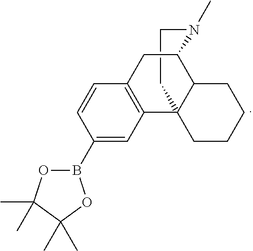

To a solution of (4bS,9S)-11-methyl-6,7,8,8a,9,10-hexahydro-5H-9,4b-(epiminoethano)phenanthren-3-yl trifluoromethanesulfonate (15.00 g, 38.6 mmol) in degassed 1,4-dioxane (450 mL) was added bis(pinacolato)diboron (14.68 g, 57.8 mmol) and potassium acetate (11.35 g, 115.6 mmol). [1,1′-Bis(diphenylphosphino)ferrocene]dichloropalladium(II) (2.82 g, 3.85 mmol) and 1,1′-Bis(diphenylphosphino)ferrocene (2.14 g, 3.85 mmol) were added and the reaction mixture heated at 90° C. for 16 hours. The reaction mixture was cooled to room temperature and the solvent was removed under reduced pressure. The residue was partitioned between ethyl acetate and water and filtered through celite. The organic layer was washed with water, dried (MgSO₄) and the solvent removed under reduced pressure. The crude material was purified by silica chromatography twice, eluted with 4% triethylamine in 1:1 dichloromethane/ethyl acetate to give (4bS,9S)-11-methyl-3-(4,4,5,5-tetramethyl-1,3,2-dioxaborolan-2-yl)-6,7,8,8a,9,10-hexahydro-5H-9,4b-(epiminoethano)phenanthrene (13.11 g, 93% yield); [M+H]⁺ 368.2, 286.2.

### Synthesis of Compound-2: Synthesis of 2-((4bS,9S)-11-methyl-6,7,8,8a,9,10-hexahydro-5H-9,4b-(epiminoethano)phenanthren-3-yl)-1,3,4-thiadiazole hydrochloride

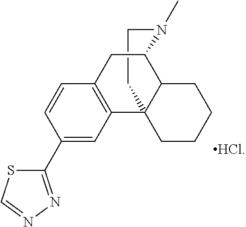

Reaction was carried out in three batches. To a solution of (4bS,9S)-11-methyl-3-(4,4,5,5-tetramethyl-1,3,2-dioxaborolan-2-yl)-6,7,8,8a,9,10-hexahydro-5H-9,4b-(epiminoethano)phenanthrene (2.0 g, 5.44 mmol) in 1,4-dioxane (60 mL) and 1M Na₂CO₃ aq (21.8 mL) was added ethyl 5-bromo-1,3,4-thiadiazole-2-carboxylate (2.6 g, 10.97 mmol) and the mixture was degassed by bubbling through argon for 10 minutes.

Tetrakis(triphenylphosphine)palladium(0) (630 mg, 0.54 mmol) was added and the reaction mixture was heated in a microwave reactor at 120° C. for 1 hour. The reaction mixture was partitioned between ethyl acetate and water and the aqueous phase was extracted with ethyl acetate. The combined organic phase was dried (MgSO₄) and the solvent removed under reduced pressure. The crude material purified by silica chromatography eluted with 5% triethylamine in 1:1 dichloromethane/ethyl acetate to give 2-((4bS,9S)-11-methyl-6,7,8,8a,9,10-hexahydro-5H-9,4b-(epiminoethano)phenanthren-3-yl)-1,3,4-thiadiazole (256 mg, 14% yield).

To 2-((4bS,9S)-11-methyl-6,7,8,8a,9,10-hexahydro-5H-9,4b-(epiminoethano)phenanthren-3-yl)-1,3,4-thiadiazole (250 mg, 0.77 mmol) in diethyl ether (2.5 mL) was added 2M HCl in diethyl ether (1.54 mL, 3.07 mmol). After 10 minutes, the solvent was decanted and the solid washed with ethyl acetate twice before drying under vacuum at 40° C. to give 2-((4bS,9S)-11-methyl-6,7,8,8a,9,10-hexahydro-5H-9,4b-(epiminoethano)phenanthren-3-yl)-1,3,4-thiadiazole hydrochloride (260 mg, 94% yield); [M+H]⁺ 326.14. ¹H NMR (300 MHz, MeOD) 9.44 (1H, s), 8.04 (1H, s), 7.84 (1H, d), 7.43 (1H, d), 3.70 (1H, br s), 3.27-3.16 (1H, m), 2.98-2.92 (3H, br s), 2.78-2.62 (2H, m), 2.13-1.91 (2H, m), 1.78-1.06 (10H, m).

### Synthesis of Compound-3

### Compound-3a: Synthesis of (4bS,9S)—N′-acetyl-11-methyl-6,7,8,8a,9,10-hexahydro-5H-9,4b-(epiminoethano)phenanthrene-3-carbohydrazide

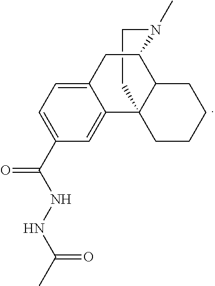 [SMILES: CC(=O)NNC(=O)c1ccc2c(c1)[C@]13CCCCC1[C@H](C2)N(C)CC3]

To a solution of (4bS,9S)-11-methyl-6,7,8,8a,9,10-hexahydro-5H-9,4b-(epiminoethano)phenanthren-3-yl trifluoromethanesulfonate (5.0 g, 12.8 mmol) in degassed dimethylsulfoxide (65 mL) was added N-hydroxysuccinimide (3.0 g, 26.1 mmol), palladium (II) acetate (0.29 g, 1.29 mmol), 4,5-Bis(diphenylphosphino)-9,9-dimethylxanthene (0.74 g, 1.29 mmol) and triethylamine (3.6 mL, 25.8 mmol). The reaction was heated to 70° C. under an atmosphere of carbon monoxide overnight. The reaction was allowed to cool to room temperature. To the reaction was added acetohydrazide (1.9 g, 25.6 mmol) and the reaction stirred at room temperature overnight. The dimethylsulfoxide was removed under reduced pressure. The residue was dissolved in dichloromethane and filtered through celite. The liquors were concentrated under reduced pressure. The crude material was purified by silica column chromatography eluted with 5% methanol in dichloromethane to 5% NH₃/methanol in dichloromethane to give (4bS,9S)—N′-acetyl-11-methyl-6,7,8,8a,9,10-hexahydro-5H-9,4b-(epiminoethano)phenanthrene-3-carbohydrazide (2.3 g, 52% yield); [M+H]⁺342.2.

Compound 3: Synthesis of 2-methyl-5-((4bS,9S)-11-methyl-6,7,8,8a,9,10-hexahydro-5H-9,4b-(epiminoethano)phenanthren-3-yl)-1,3,4-thiadiazole hydrochloride

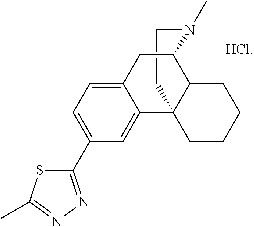 [SMILES: Cc1nnc(-c2ccc3c(c2)[C@]24CCCCC2[C@H](C3)N(C)CC4)s1]

To a solution of (4bS,9S)—N′-acetyl-11-methyl-6,7,8,8a,9,10-hexahydro-5H-9,4b-(epiminoethano)phenanthrene-3-carbohydrazide (1.1 g, 3.2 mmol) in tetrahydrofuran (40 mL) was added Lawesson's reagent (2.6 g, 6.4 mmol). The reaction was heated to 50° C. overnight. The reaction was cooled to room temperature before the solvent was removed under reduced pressure. The residue was dissolved in dichloromethane and purified by silica chromatography eluted with 0-10% NH₃/methanol in dichloromethane. The material was further purified by preparative HPLC to give 2-methyl-5-((4bS,9S)-11-methyl-6,7,8,8a,9,10-hexahydro-5H-9,4b-(epiminoethano)phenanthren-3-yl)-1,3,4-thiadiazole (0.54 g, 50% yield).

To a solution of 2-methyl-5-((4bS,9S)-11-methyl-6,7,8,8a,9,10-hexahydro-5H-9,4b-(epiminoethano)phenanthren-3-yl)-1,3,4-thiadiazole (0.54 g, 1.59 mmol) in ethyl acetate (30 mL) was added 4M HCl in diethyl ether (4 mL, 16.0 mmol). The reaction was stirred for 2 hours before the solvent was removed under reduced pressure. The resulting solid was dried under vacuum to give 2-methyl-5-((4bS,9S)-11-methyl-6,7,8,8a,9,10-hexahydro-5H-9,4b-(epiminoethano)phenanthren-3-yl)-1,3,4-thiadiazole hydrochloride (0.59 g, 99% yield); [M+H]⁺ 340.35. ¹H NMR (300 MHz, d₆-DMSO) 11.06 (1H, s), 7.82 (1H, s), 7.72 (1H, d), 7.35 (1H, d), 3.35-2.82 (4H, m), 2.80-2.67 (6H, m), 2.44-2.31 (1H, m), 2.22 (1H, d), 2.06-1.85 (1H, m), 1.65-1.18 (7H, m), 1.17-0.99 (1H, m), 0.98-0.76 (1H, m).

### Biological Examples

In-Vitro FLIPR Ca++Flux Data (NR1/NR2A Expressing HEK Cells)

The NMDA receptor is an ionotropic glutamate receptor that forms a cation-selective channel. Test articles were assessed for their ability to inhibit calcium flux produced by a positive control agonist (100 μM Glutamic acid+20 μM Glycine) in cloned human NMDA receptor (NR1/NR2A) channel (encoded by the GRIN1 and GRIN2A genes) expressing HEK293 cells. A Fluo-8 calcium kit and a Fluorescence Imaging Plate Reader (FLIPRTETRA™) instrument was used to measure calcium flux. The potential antagonist effect of each test article was measured at eight concentrations (0.1, 0.3, 1, 3, 10, 30, 100, 300 μM; n=4 per concentration) after activating NR1/NR2A channel with the positive control agonist (100 μM Glutamic acid+20 μM Glycine). The ability of each test article to inhibit this signal was assessed and compared to the positive control antagonist, (+) MK-801. The signal, elicited in the presence of the positive control agonist (Glutamic acid+Glycine), was set to 100 (0% inhibition) and the signal in the presence of the positive control antagonist (Glutamic acid+Glycine+100 μM (+) MK-801) was set to 0 (100% inhibition). Normalized % inhibition vs log concentration data was fit to a four parameter dose response in GraphPad Prism with the top of the curve constrained to 100% in order to calculate the half maximal inhibitory concentration (IC₅₀). Table 2 compares the effects of multiple test compounds to known reference compounds in this assay.

TABLE 2

| | IC₅₀ NMDA |
|---|---|
| Compound | (μM) |
| Dizocilpine (MK-801) | 1.2 |
| Phencyclidine (PCP) | 7.5 |
| Ketamine | 57.2 |
| Dextromethorphan (DMX) | 106.8 |
| 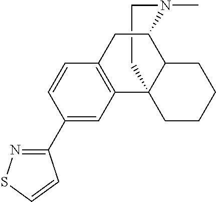 [SMILES: CN1CC[C@@]23CCCCC2[C@@H]1Cc1ccc(-c2ccsn2)cc13] | 15 |
| 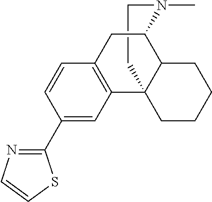 [SMILES: CN1CC[C@@]23CCCCC2[C@@H]1Cc1ccc(-c2nccs2)cc13] | 33 |
| 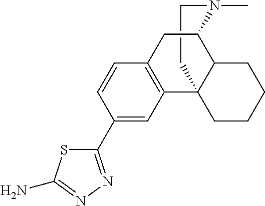 [SMILES: CN1CC[C@@]23CCCCC2[C@@H]1Cc1ccc(-c2nnc(N)s2)cc13] | 35.5 |
| 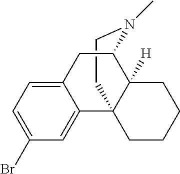 [SMILES: CN1CC[C@@]23CCCC[C@@H]2[C@@H]1Cc1ccc(Br)cc13] | 49 |
| 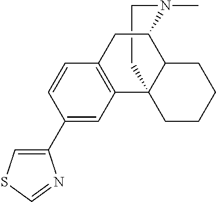 [SMILES: CN1CC[C@@]23CCCCC2[C@@H]1Cc1ccc(-c2cscn2)cc13] | 50 |
| 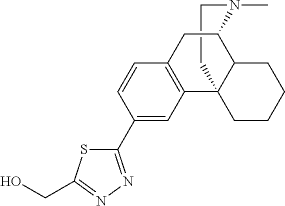 [SMILES: CN1CC[C@@]23CCCCC2[C@@H]1Cc1ccc(-c2nnc(CO)s2)cc13] | 47.5 |
| Compound-3 | 94 |
| Compound-1 | 225 |
| Compound-2 | 225 |
| 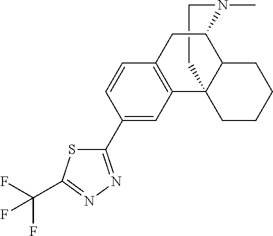 [SMILES: CN1CC[C@@]23CCCCC2[C@@H]1Cc1ccc(-c2nnc(C(F)(F)F)s2)cc13] | >500 |
| 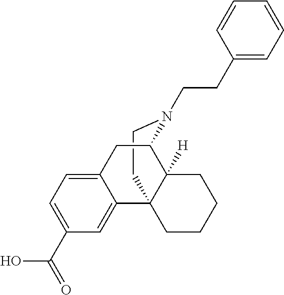 [SMILES: O=C(O)c1ccc2c(c1)[C@]13CCCC[C@@H]1[C@H](C2)N(CCc1ccccc1)CC3] | >500 |
| 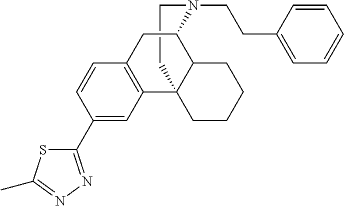 [SMILES: Cc1nnc(-c2ccc3c(c2)[C@]24CCCCC2[C@H](C3)N(CCc2ccccc2)CC4)s1] | >500 |
| 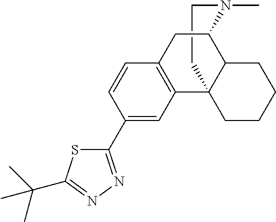 [SMILES: CN1CC[C@@]23CCCCC2[C@@H]1Cc1ccc(-c2nnc(C(C)(C)C)s2)cc13] | >500 |
| 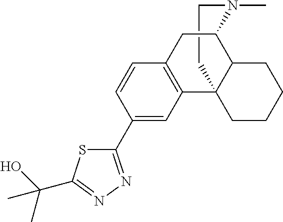 [SMILES: CN1CC[C@@]23CCCCC2[C@@H]1Cc1ccc(-c2nnc(C(C)(C)O)s2)cc13] | >500 |
| 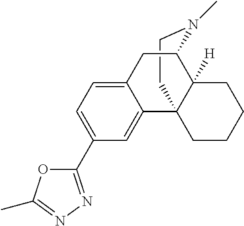 [SMILES: Cc1nnc(-c2ccc3c(c2)[C@]24CCCC[C@@H]2[C@H](C3)N(C)CC4)o1] | >500 |

### In-Vitro FLIPR Ca++Flux Assay in NR1/2a Expressing HEK Cells

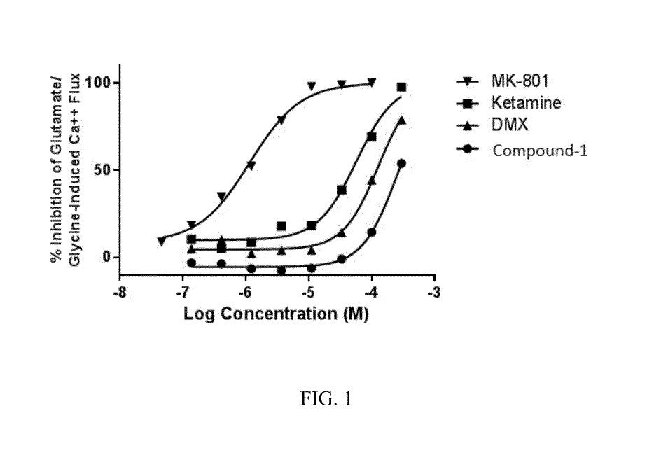

The NMDA receptor is an ionotropic glutamate receptor that forms a cation-selective channel. Test articles were assessed for their ability to inhibit calcium flux produced by a positive control agonist (100 μM Glutamic acid+20 μM Glycine) in cloned human NMDA receptor (NR1/NR2A) channel (encoded by the GRIN1 and GRIN2A genes) expressing HEK293 cells. A Fluo-8 calcium kit and a Fluorescence Imaging Plate Reader (FLIPRTETRA™) instrument was used to measure calcium flux. The potential antagonist effect of each test article was measured at eight concentrations (0.1, 0.3, 1, 3, 10, 30, 100, 300 μM; n=4 per concentration) after activating NR1/NR2A channel with the positive control agonist (100 μM Glutamic acid+20 μM Glycine). The ability of each test article to inhibit this signal was assessed and compared to the positive control antagonist, (+) MK-801. The signal, elicited in the presence of the positive control agonist (Glutamic acid+Glycine), was set to 100 (0% inhibition) and the signal in the presence of the positive control antagonist (Glutamic acid+Glycine+100 μM (+) MK-801) was set to 0 (100% inhibition). Normalized % inhibition vs. log concentration data was fit to a four parameter dose response in GraphPad Prism with the top of the curve constrained to 100% in order to calculate the half maximal inhibitory concentration (IC₅₀). Results of the study comparing Compound-1 to reference compounds is provided in FIG. 1 and concludes that all compounds displayed antagonist activity. The potency of Compound-1 was less than the reference compounds, but it demonstrated good efficacy in this assay system. The lower observed potency is consistent with the lower binding affinity of Compound-1 for the PCP Binding site. Collectively, these traits support the conclusion that Compound-1 is a low-trapping NMDA antagonist.

### CEREP NMDA PCP Site Binding Study

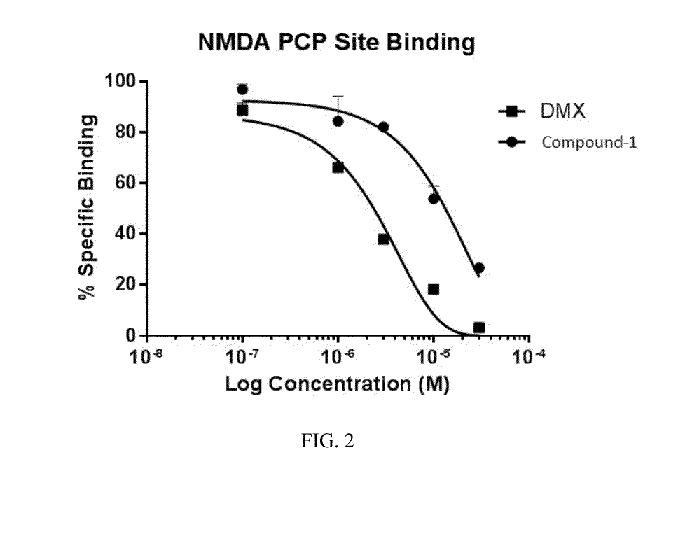

Test articles were assessed for their ability to inhibit specific binding of phencyclidine to the PCP binding site of NMDA receptors in rat cerebral cortex cells (CEREP PCP antagonist radioligand assay #0124). The specific ligand binding to the receptors is defined as the difference between the total binding and the nonspecific binding determined in the presence of an excess of unlabelled ligand. Phencyclidine radioligand was used at a concentration of 10 nM. The effect of each test article was measured at five concentrations (0.03, 0.1, 0.3, 1, 10 μM). Scintillation counting of radioligand was performed. In each experiment, a reference compound (MK-801) was tested concurrently with the test compounds, and the data were compared with historical values determined at Cerep to confirm assay performance. The IC50 values (concentration causing a half-maximal inhibition of control specific binding) and Hill coefficients (nH) were determined by non-linear regression analysis of the competition curves generated with mean replicate values using Hill equation curve fitting (Y=D+[(A−D)/(1+(C/C50)nH)], where Y=specific binding, D=minimum specific binding, A=maximum specific binding, C=compound concentration, C50=IC50, and nH=slope factor). This analysis was performed using a software developed at Cerep (Hill software) and validated by comparison with data generated by the commercial software SIGMAPLOT® 4.0 for WINDOWS® (© 1997 by SPSS Inc.). The inhibition constants (Ki) were calculated using the Cheng Prusoff equation (Ki=IC50/(1+(L/KD)), where L=concentration of radioligand in the assay, and KD=affinity of the radioligand for the receptor). A scatchard plot is used to determine the Ki of Compound-1 to reference compound (DMX). Results of the study, as provided in FIG. 2, conclude that Compound-1 (Ki=7.4 μM) binds competitively to the PCP binding site, while reference compound DMX binds at a Ki=1.3 μM. While the Ki of Compound-1 is higher (denoting lower binding affinity) than the known reference (DMX), this is a desirable characteristic. Compounds that bind with high affinity to the PCP-binding site are known to become “trapped” within the ion channel. There is a good correlation between binding affinity for the PCP site and the degree of trapping; high affinity compounds are trapped at higher rates. Drugs described as high-trapping have been shown to have an undesirable side effect profile, while low-trapping drugs have improved safety. Consequently, Compound-1 is expected to have a better safety profile than other drugs binding to the PCP-binding site with higher affinity.

### Efficacy Study in an Animal Model of Agitation

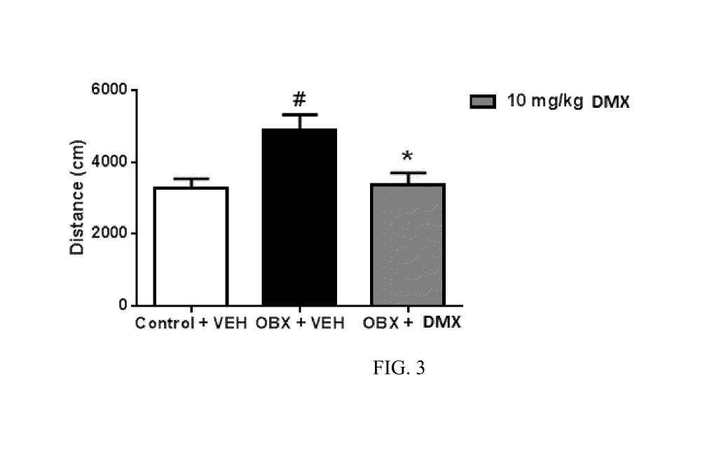

The objective of this study is to assess the efficacy of Compound-1 in an animal model of agitation: the olfactory bulbectomized (OBX) rat. Removal of the olfactory bulbs in rodents significantly increases locomotor activity and stereotypy, behaviors that are associated with agitation. FIG. 3 demonstrates that dextromethorphan, i.e., DEX, (a comparator compound) blocks the locomotor response in OBX rats in the Open Field arena.

### Experimental Design

The study design included 4 treatment groups. Rats were assigned to their respective treatments by stratifying into groups based on body weight and arena. The experiment was carried out on a single day.

TABLE 3

| | | | | Solution | | Injection | |
|---|---|---|---|---|---|---|---|
| | | | | Conc. | Dose | Volume | Final Dose |
| Group | N | Surgery | Compound | (mg/mL) | Frequency | (mL/kg) | (mg/kg) |
| A | 8 | Control | Compound-1 | 0 | Once | 1.0 | 0 |
| B | 12 | OBX | Compound-1 | 0 | Once | 1.0 | 0 |
| C | 12 | OBX | Compound-1 | 10 | Once | 1.0 | 10 |
| D | 12 | OBX | Compound-1 | 30 | Once | 1.0 | 30 |

Forty-four male Sprague Dawley rats (Charles River, Raleigh, N.C.) were used for this study. Rats were single housed and weighed 200-225 g upon receipt. All rats were maintained on a 12 h light-12 h dark cycle with free access to food and water. Husbandry was conducted in accordance with the 2011 Guide for the Care and Use of Laboratory Animals (NRC, 2011).

Subcutaneous doses were administered via flank injection at a dose volume of 1 mL/kg 30 minutes before behavioral testing. Dose volumes were adjusted for the weight of the animal on the day of the experiment.

OBX rats and their weight-matched non-surgery controls were allowed to acclimate/recover from the surgical procedure for at least 4 weeks to permit the onset of the behavioral deficit. Rats were handled periodically throughout this acclimation period to prevent excessive aggression. Rats were placed in the open field arena 30 minutes post-drug administration for a 30 minute test. This experiment was run under dim light and low white noise conditions.

The following parameters were assayed:
1. Distance travelled in first 5 mins (cm) to assess locomotor activity in OBX versus control rats.
2. Distance travelled in 30 min test (cm) to assess drug efficacy.
   The behavior was recorded and analyzed by an automated tracking system (Med Associates). GraphPad Prism 6.0 was used to prepare graphs and to perform data analysis including one-way ANOVA or t-test analysis of locomotor activity where appropriate.

### Results:

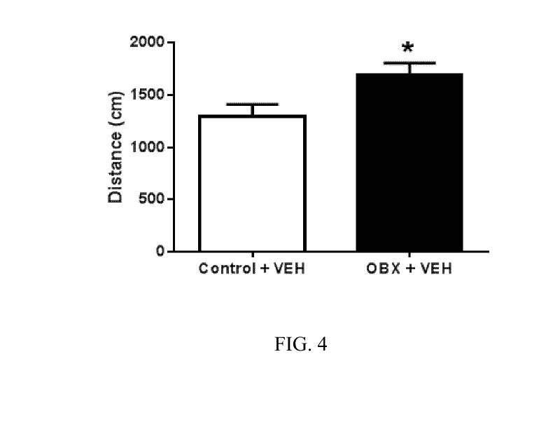

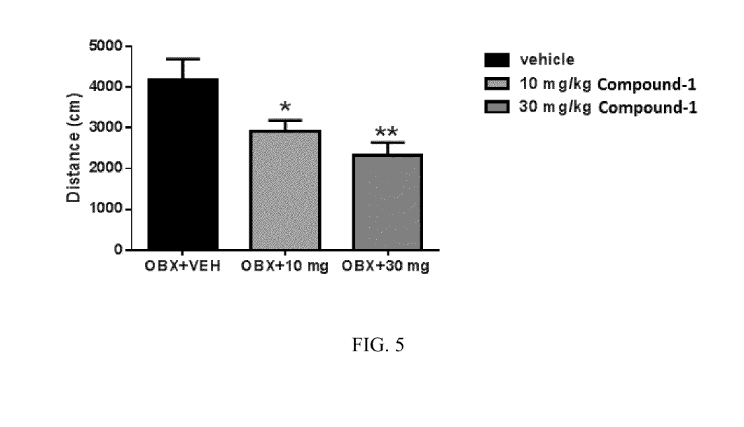

OBX rats demonstrated a significant increase in locomotor activity compared to non-surgery controls (two-tailed t-test, t=2.437 df=14; p<0.05). See FIG. 4. After a single subcutaneous injection, Compound-1 blocked this hyperlocomotor response (One-way ANOVA: main effect observed F_{(2,32)}=6.598 p<0.01), an effect that appeared to be dose-dependent (Dunnett's post-hoc analysis: p<0.05; p<0.01). See FIG. 5. Compound-1 decreased agitation-like behavior in the OBX rat.

### Efficacy Study in an Animal Model of Anxiety

Removal of the olfactory bulbs in rodents significantly increases anxiety-like behaviors. Many rodents exhibit burying behavior, commonly referred to as “defensive burying” in response to aversive stimuli, e.g., predatory animals or shock prod. Rodents will also bury non-aversive unconditioned objects, such as food pellets and glass marbles; an effect reversible by compounds that have anxiolytic efficacy. The objective of this study is to assess the efficacy of Compound-1 and comparator drug dextromethorphan to attenuate burying behavior in the OBX rat.

Experimental Design

The study design included 4 treatment groups. Rats were assigned to their respective treatments by stratifying into groups based on body weight and arena. The experiment was carried out on a single day.

TABLE 4

| | | | | Solution | | Injection | Final |
|---|---|---|---|---|---|---|---|
| | | | | Conc. | Dose | Volume | Dose |
| Group | N | Surgery | Compound | (mg/mL) | Frequency | (mL/kg) | (mg/kg) |
| A | 8 | Control | Compound-1 | 0 | Once | 1.0 | 0 |
| B | 8 | OBX | Compound-1 | 0 | Once | 1.0 | 0 |
| C | 8 | OBX | Compound-1 | 10 | Once | 1.0 | 10 |
| D | 8 | OBX | dextromethorphan | 10 | Once | 1.0 | 10 |

Thirty-two male Sprague Dawley rats (Charles River, Raleigh, N.C.) were used for this study. Rats were single housed and weighed 200-225 g upon receipt. All rats were maintained on a 12 h light-12 h dark cycle with free access to food and water. Husbandry was conducted in accordance with the 2011 Guide for the Care and Use of Laboratory Animals (NRC, 2011).

Subcutaneous doses were administered via flank injection at a dose volume of 1 mL/kg 30 minutes before behavioral testing. Dose volumes were adjusted for the weight of the animal on the day of the experiment.

OBX rats and their weight-matched non-surgery controls were allowed to acclimate/recover from the surgical procedure for at least 4 weeks to permit the onset of the behavioral deficit. Rats were handled periodically throughout this acclimation period to prevent excessive aggression. Rats were injected and placed in the marble burying arena (20 evenly spaced glass marbles; 20 min test) 30 minutes post-drug administration. This experiment was run under bright light and white noise conditions.

The following parameters were assayed:

Marbles were considered “buried” if more than ⅔ surface was not visible. The number of uncovered/buried marbles was recorded and photographed by experimenter blind to drug treatment. GraphPad Prism 6.0 was used to prepare graphs and to perform data analysis including one-way ANOVA or t-test analysis of data where appropriate.

### Results:

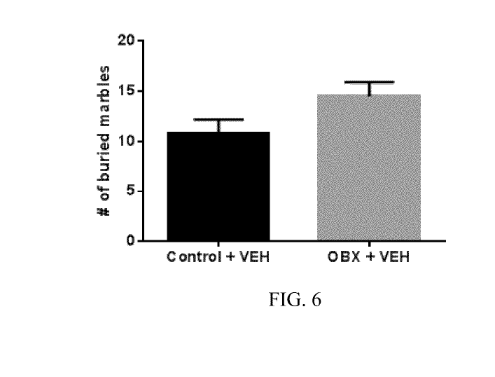

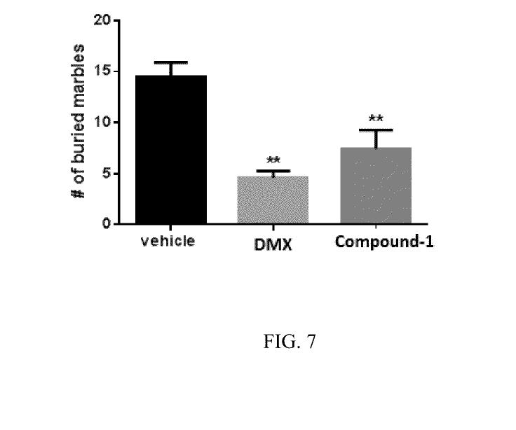

OBX rats buried more marbles compared to non-surgery controls (two-tailed t-test, t=1.867 df=13; p=0.08). See FIG. 6. After a single subcutaneous injection, both Compound-1 and dextromethorphan blocked this burying response (One-way ANOVA: main effect observed F_{(2,21)}=13.67 p<0.001; Dunnett's post-hoc analysis: p<0.01). See FIG. 7. Compound-1 decreased anxiety-like behavior in the OBX rat. The effect was comparable to dextromethorphan in this assay.

### Study of the Effect of Compound-1 on Locomotor Activity in Rats

Significant changes in locomotor activity may indicate an abnormal response to a drug (e.g., psychomotor retardation, agitation or behavioral sensitization). The Open field test is a commonly used qualitative and quantitative measure of general locomotor activity and willingness to explore in rodents. The objective of this study is to evaluate the effect of Compound-1 on locomotor activity in the (non-olfactory bulbectomized) rat. This study serves as a control experiment for the agitation and anxiety assays described above.

### Experimental Design

The study design included 2 treatment groups. Rats were randomized into their respective treatments. The experiment was carried out on a single day.

TABLE 5

| | | | Solution | | Injection | Final |
|---|---|---|---|---|---|---|
| | | | Conc. | Dose | Volume | Dose |
| Group | N | Compound | (mg/mL) | Frequency | (mL/kg) | (mg/kg) |
| A | 8 | Compound-1 | 0 | Once | 1.0 | 0 |
| B | 8 | Compound-1 | 10 | Once | 1.0 | 10 |

Sixteen male Sprague Dawley rats (Charles River, Raleigh, N.C.) were used for this study. Rats were single housed and weighed 200-225 g upon receipt. All rats were maintained on a 12 h light-12 h dark cycle with free access to food and water. Husbandry was conducted in accordance with the 2011 Guide for the Care and Use of Laboratory Animals (NRC, 2011).

Subcutaneous doses were administered via flank injection at a dose volume of 1 mL/kg 30 minutes before behavioral testing. Dose volumes were adjusted for the weight of the animal on the day of the experiment.

Rats were allowed to acclimate to the facility for 1 week before behavioral testing. Rats were placed in the Open field arena 30 minutes post-drug administration for a 30 min test. This experiment was run under dim light and low white noise conditions.

The following parameters were assayed:

1. Distance travelled (cm)

2. Number of jumps

3. Stereotypy counts (arbitrary unit)

4. Number of rears (exploratory behavior)

5. Average velocity (cm/s)

The behavior was recorded and analyzed by an automated tracking system (Med Associates). GraphPad Prism 6.0 was used to prepare graphs and to perform data analysis including one-way ANOVA or t-test analysis of locomotor activity where appropriate.

### Results:

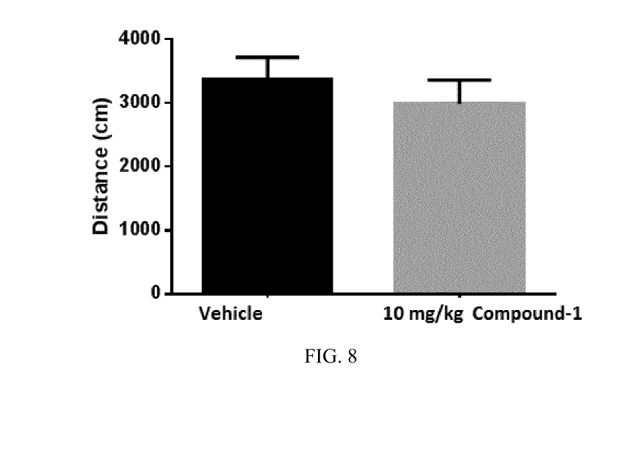

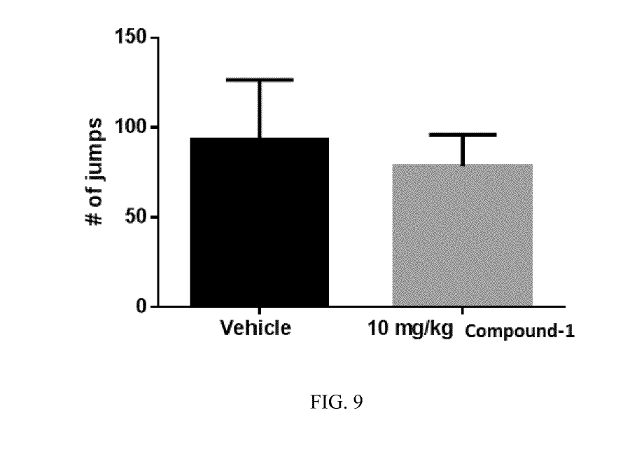

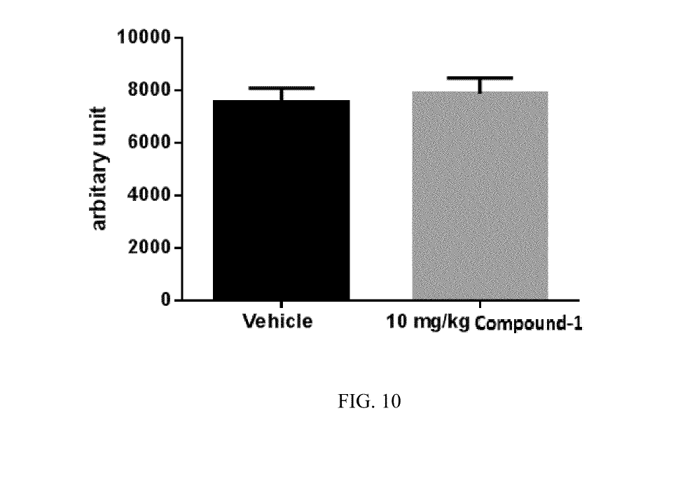

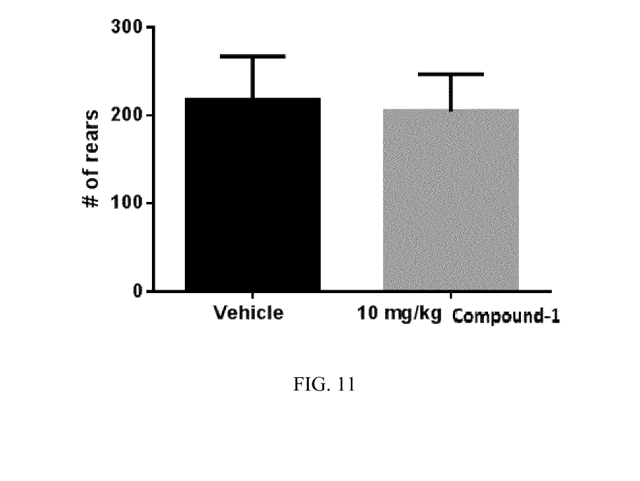

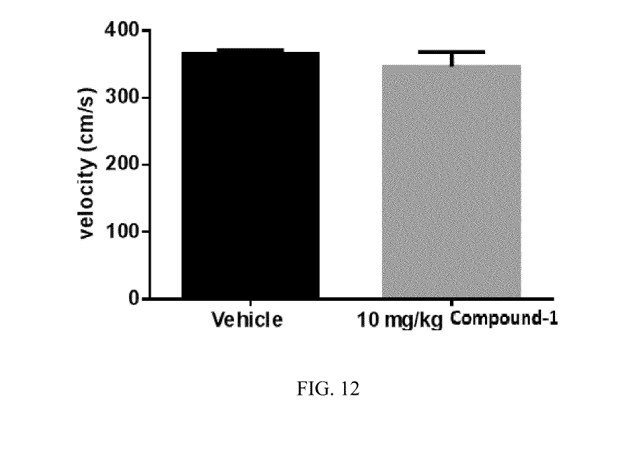

Rats treated with Compound-1 had no effect on locomotor activity (t=0.7536; df=14; p=0.5; FIG. 8), jumping behavior (t=0.3930; df=14; p=0.7; FIG. 9), stereotypy (t=0.3895; df=14; p=0.7; FIG. 10), rearing (t=0.2039; df=14; p=0.8; FIG. 11), or average velocity (t=0.8295; df=14; p=0.4; FIG. 12) compared to vehicle treated controls (two-tailed t-tests). Compound-1 did not significantly alter locomotor behavior in Sprague Dawley rats.

While this invention has been particularly shown and described with references to preferred embodiments thereof, it will be understood by those skilled in the art that various changes in form and details may be made therein without departing from the scope of the invention encompassed by the appended claims.

1. A method of alleviating a neuropsychiatric behavioral symptom associated with Alzheimer's disease comprising administering to a subject in need thereof an effective amount of a compound of formula: 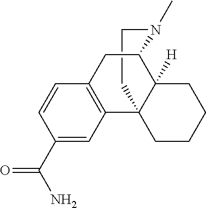 [SMILES: CN1CC[C@@]23CCCC[C@@H]2[C@@H]1Cc1ccc(C(N)=O)cc13]
or a pharmaceutically acceptable salt thereof.

2. The method according to claim 1, wherein the behavioral symptom being alleviated is selected from the group consisting of anxiety, agitation, aggression, depression, hallucination, memory loss, confusion, repetition, sleep issues, sun downing, suspicion, delusions, and wandering.

3. The method according to claim 2, wherein the behavioral symptom being alleviated is anxiety.

4. The method according to claim 2, wherein the behavioral symptom being alleviated is agitation.

5. The method according to claim 2, wherein the behavioral symptom being alleviated is aggression.

6. The method according to claim 1, wherein said compound is administered in a daily dose of about 1 mg to about 300 mg/day.

7. The method of claim 6, wherein said daily dose is administered orally.

8. A method of alleviating a neuropsychiatric behavioral symptom associated with Alzheimer's disease, comprising administering a low trapping N-methyl-D-aspartate (NMDA) channel blocker to a subject in need thereof, wherein the binding affinity (Ki) range is 1-100 μM to the PCP binding site of the NMDA receptor, and the IC₅₀ range is 30 to 300 μM in a FLIPR calcium flux assay, wherein the method does not cause psychotomimetic liability or dissociative side effects.

9. The method of claim 8, wherein the IC₅₀ range is 100 to 300 μM.

10. The method of claim 9, wherein the IC₅₀ range is 150 to 300 μM.

11. The method of claim 8, wherein the binding affinity (Ki) range is 5-50 μM.

12. The method of claim 11, wherein the binding affinity (Ki) range is 10-25 μM.

13. The method of claim 8, wherein the psychotomimetic liability is visual hallucinations.

14. The method of claim 8, wherein the dissociative side effects comprise feeling abnormal, disinhibition, illusion and dissociation.

15. The method of claim 8, wherein the low trapping NMDA channel blocker is a compound of formula: 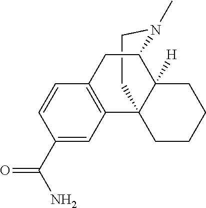 [SMILES: CN1CC[C@@]23CCCC[C@@H]2[C@@H]1Cc1ccc(C(N)=O)cc13]
or a pharmaceutically acceptable salt thereof.

16. A method of treating a disease mediated by an NMDA receptor, comprising administering a low trapping N-methyl-D-aspartate (NMDA) channel blocker to a subject in need thereof, wherein the binding affinity (Ki) range is 1-100 μM to the PCP binding site of the NMDA receptor, and the IC₅₀ range is 30 to 300 μM in a FLIPR calcium flux assay, wherein the method provides an improved therapeutic window with lower neural toxicity.

17. The method of claim 16, wherein the IC₅₀ range is 100 to 300 μM.

18. The method of claim 17, wherein the IC₅₀ range is 150 to 300 μM.

19. The method of claim 16, wherein the binding affinity (Ki) range is 5-50 μM.

20. The method of claim 19, wherein the binding affinity (Ki) range is 10-25 μM.

21. The method of claim 16, wherein the low trapping NMDA channel blocker is a compound of formula: 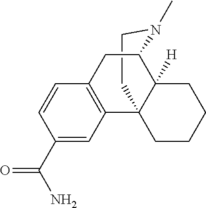 [SMILES: CN1CC[C@@]23CCCC[C@@H]2[C@@H]1Cc1ccc(C(N)=O)cc13]
or a pharmaceutically acceptable salt thereof.

22. A method of alleviating a neuropsychiatric behavioral symptom associated with Alzheimer's disease comprising administering to a subject in need thereof an effective amount of a compound of Formula I or a pharmaceutically acceptable salt thereof: 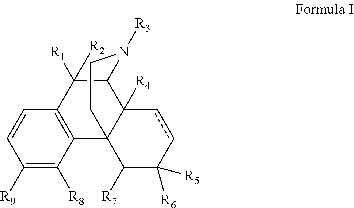
wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₅ and R₆ together form a carbonyl or a vinyl substituent;
independently R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group; and
R₉ is selected from —C(O)N(R₂₀)(R₂₁), —C(S)N(R₂₀)(R₂₁), aryl, substituted aryl, heteroaryl, substituted heteroaryl, heterocyclyl and substituted heterocyclyl.

23. A method of alleviating a neuropsychiatric behavioral symptom associated with Alzheimer's disease comprising administering to a subject in need thereof an effective amount of a compound of Formula IA or a pharmaceutically acceptable salt thereof: 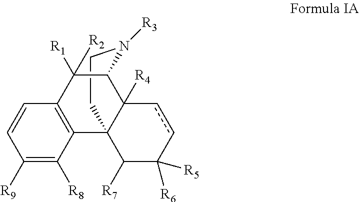
wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₅ and R₆ together form a carbonyl or a vinyl substituent;
each R₂₀ and R₂₁ is independently selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group; and,
R₉ is selected from —C(O)N(R₂₀)(R₂₁), —C(S)N(R₂₀)(R₂₁), aryl, substituted aryl, heteroaryl, substituted heteroaryl, heterocyclyl and substituted heterocyclyl.

24. A method of alleviating a neuropsychiatric behavioral symptom associated with Alzheimer's disease comprising administering to a subject in need thereof an effective amount of a compound of Formula IB or a pharmaceutically acceptable salt thereof: 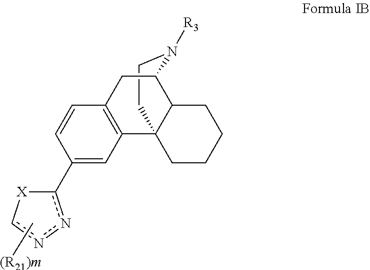
wherein, [symbol] represents a single or double bond;
m is 0, 1, 2 or 3;
X is CH₂, NR₂₀, S, S(O)₂ or O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; and
R₂₁ is selected from hydrogen, halogen, aliphatic, substituted aliphatic, aryl and substituted aryl.

25. A compound of Formula IB or a pharmaceutically acceptable salt thereof: 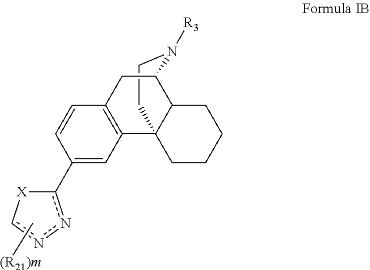
wherein, [symbol] represents a single or double bond;
m is 0, 1, 2 or 3;
X is CH₂, NR₂₀, S, S(O)₂ or O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; and
R₂₁ is selected from hydrogen, halogen, aliphatic, substituted aliphatic, aryl and substituted aryl.

26. A compound according to claim 25, wherein R₂₁ is —CH₃ and m is 1.

27. A compound according to claim 25, wherein X is S.

28. A compound selected from Compound 2 or 3, or a pharmaceutically acceptable salt thereof:

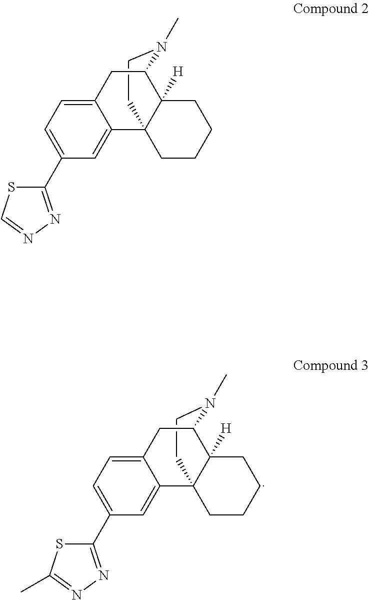 [SMILES: CN1CC[C@@]23CCCC[C@@H]2[C@@H]1Cc1ccc(-c2nncs2)cc13 ; Cc1nnc(-c2ccc3c(c2)[C@]24CCCC[C@@H]2[C@H](C3)N(C)CC4)s1]

29. A composition comprising a compound according to claim 25 and a pharmaceutically acceptable carrier.

30. A method of treating a disease or disorder, or a symptom of a disease or disorder mediated by an N-methyl-D-aspartate (NMDA) receptor comprising the step of administering a compound of Formula I, or a pharmaceutically acceptable salt thereof, to a subject in need thereof: 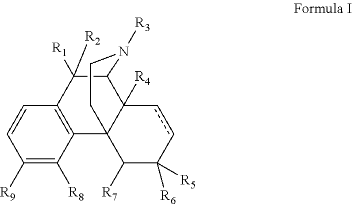
wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
Alternatively R₅ and R₆ together form a carbonyl or a vinyl substituent;
each R₂₀ and R₂₁ is independently selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group; and,
R₉ is selected from —C(O)N(R₂₀)(R₂₁), —C(S)N(R₂₀)(R₂₁), aryl, substituted aryl, heteroaryl, substituted heteroaryl, heterocycyl and substituted heterocyclyl.

31. A method of treating a disease or disorder, or a symptom of a disease or disorder mediated by an N-methyl-D-aspartate (NMDA) receptor comprising the step of administering a compound of Formula IA, or a pharmaceutically acceptable salt thereof, to a subject in need thereof: 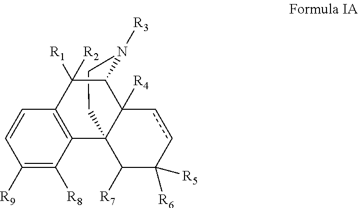
wherein, [symbol] represents a single or double bond;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
Alternatively, R₅ and R₆ together form a carbonyl or a vinyl substituent;
each R₂₀ and R₂₁ is independently selected from hydrogen, halogen, aliphatic, substituted aliphatic, aryl or substituted aryl;
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group; and,
R₉ is selected from —C(O)N(R₂₀)(R₂₁), —C(S)N(R₂₀)(R₂₁), aryl, substituted aryl, heteroaryl, substituted heteroaryl, heterocycyl and substituted heterocyclyl.

32. A method of treating a disease or disorder, or a symptom of a disease or disorder mediated by an N-methyl-D-aspartate (NMDA) receptor comprising the step of administering a compound of Formula IB, or a pharmaceutically acceptable salt thereof, to a subject in need thereof: 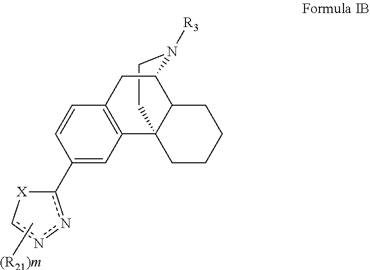
wherein, [symbol] represents a single or double bond;
m is 0, 1, 2 or 3;
X is CH₂, NR₂₀, S, S(O)₂ or O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl; and,
R₂₁ is selected from hydrogen, halogen, aliphatic, substituted aliphatic, aryl and substituted aryl.

33. A method of treating a disease or disorder, or a symptom of a disease or disorder mediated by an N-methyl-D-aspartate (NMDA) receptor comprising the step of administering a compound selected from: 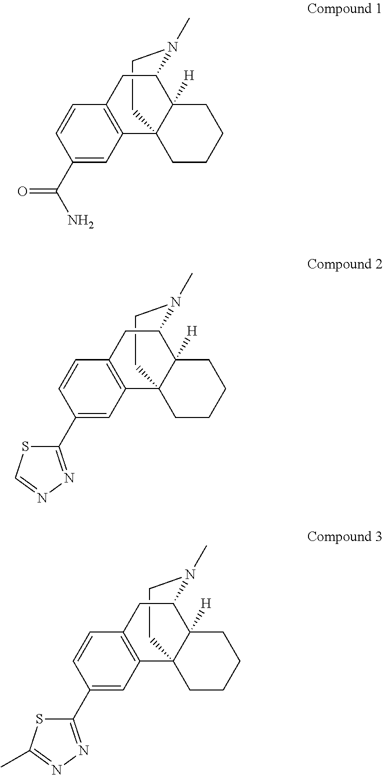 [SMILES: CN1CC[C@@]23CCCC[C@@H]2[C@@H]1Cc1ccc(C(N)=O)cc13 ; CN1CC[C@@]23CCCC[C@@H]2[C@@H]1Cc1ccc(-c2nncs2)cc13 ; Cc1nnc(-c2ccc3c(c2)[C@]24CCCC[C@@H]2[C@H](C3)N(C)CC4)s1]
or a pharmaceutically acceptable salt thereof, to a subject in need thereof.

34. The method according to claim 30, wherein said NMDA receptor mediated disease or disorder is selected from pseudobulbar affect (PBA), Alzheimer's disease, amyotrophic lateral sclerosis (ALS), Parkinson's disease, Huntington's disease, stroke, ischemia, CNS trauma, hypoxia, hypoglycemia, multiple sclerosis (MS), epilepsy, cerebral palsy (periventricular leukomalacia), spinal cord injury, chronic and severe mood disorders, acute and treatment resistant schizophrenia, bipolar depression, treatment resistant depression, major depressive disorder, obsessive compulsive disorder (OCD), drug addiction and other addictive behaviors, anxiety disorder, autism spectrum disorders, adult autism, pain management and suicidal ideation.

35. The method according to claim 30, wherein said NMDA receptor mediated disease or disorder is major depressive disorder.

36. The method according to claim 30, wherein said NMDA receptor mediated disease or disorder is Alzheimer's disease.

37. The method according to claim 30, wherein said NMDA receptor mediated disease or disorder is dementia.

38. A method of treating a disease or disorder, or a symptom of a disease or disorder mediated by an N-methyl-D-aspartate (NMDA) receptor comprising the step of administering Compound-1, or a pharmaceutically acceptable salt thereof, to a subject in need thereof:

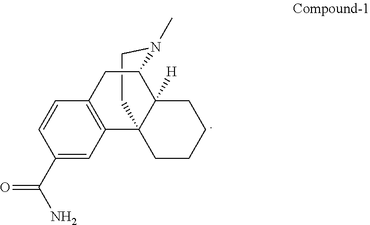 [SMILES: CN1CC[C@@]23CCCC[C@@H]2[C@@H]1Cc1ccc(C(N)=O)cc13]

39. The method according to claim 38, wherein said NMDA receptor mediated disease or disorder is major depressive disorder.

40. The method according to claim 38, wherein said NMDA receptor mediated disease or disorder is Alzheimer's disease.

41. The method according to claim 38, wherein said NMDA receptor mediated disease or disorder is dementia.

42. The method according to claim 22, wherein said compound of Formula I is selected from: 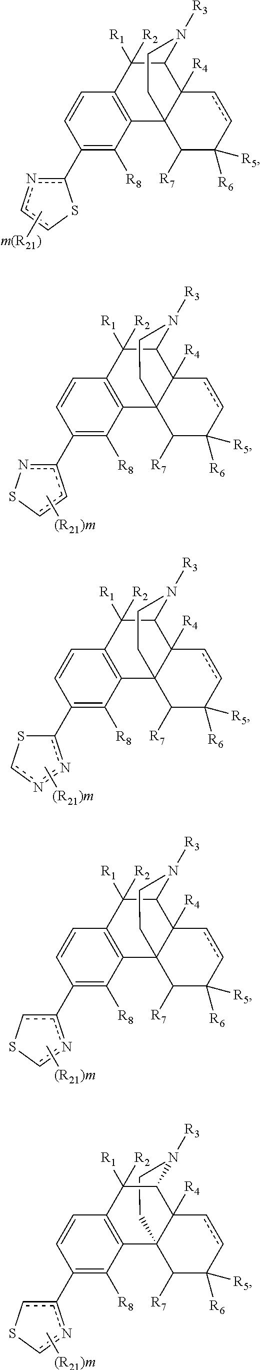 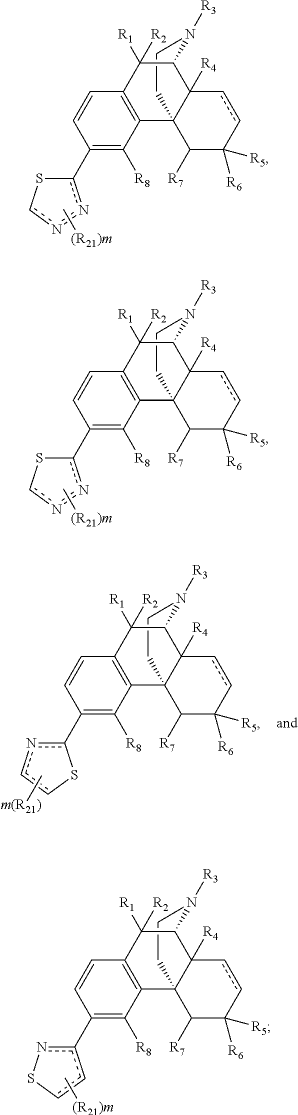
or a pharmaceutically acceptable salt thereof;
wherein, [symbol] represents a single or double bond;
m is 0, 1, 2 or 3;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₅ and R₆ together form a carbonyl or a vinyl substituent;
each R₂₀ and R₂₁ is independently selected from hydrogen, halogen, aliphatic, substituted aliphatic, aryl or substituted aryl; alternatively, two R₂₀ groups or one R₂₀ and one R₂₁ groups together with the atoms to which they are attached may form a two, three, four, five, six or seven membered ring; and,
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group.

43. The method according to claim 22, wherein said subject is administered an additional pharmaceutical agent selected from cholinesterase inhibitors and memantine, or a combination thereof.

44. The method according to claim 43, wherein said cholinesterase inhibitor is selected from the group consisting of tacrine, donepezil, rivastigmine, galantamine, and pharmaceutically acceptable salts thereof. In one embodiment, the second pharmaceutical agent is selected from acamprosate, amlodipine, argatroban, baclofen, cilostazol, cinacalcet, clopidogrel, dyphylline, fenoldopam, leflunomide, mepacrine, methimazole, phenformin, prilocaine, rifabutin, sulfisoxazole, tadalafil, terbinafine, torasemide, cinnarizine, ciclopirox, eplerenone, carbenoxolone, sulodexide, carbamazine, amobarbital, cefotetan, erythrityl tetranitrate, methyclothiazide, risedronate, enprofylline, oxtriphylline, paramethadione, cefmenoxime, aprindine, etomidate, mitiglinide, benidipine, levosimendan, and zonisamide.

45. The method according to claim 30, wherein said compound of Formula I is selected from: 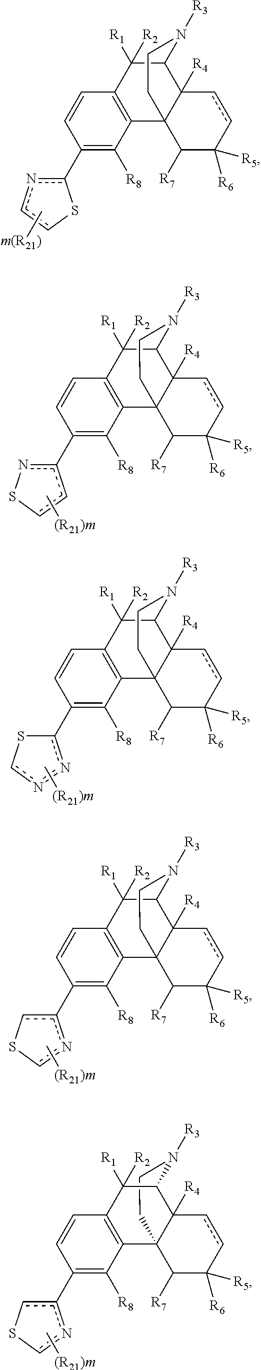 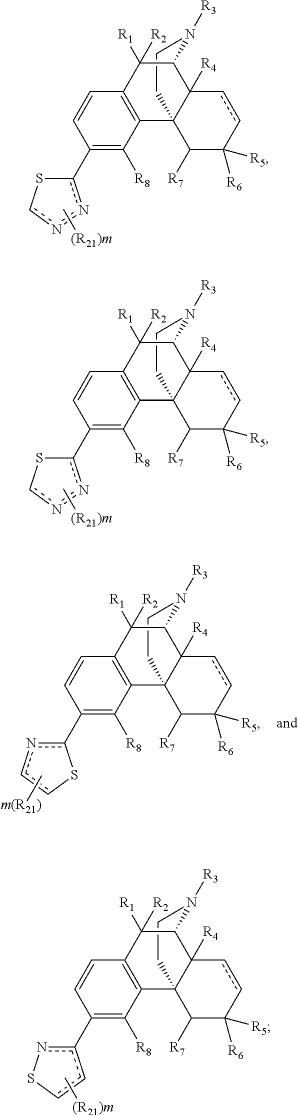
or a pharmaceutically acceptable salt thereof;
wherein, [symbol] represents a single or double bond;
m is 0, 1, 2 or 3;
R₁ and R₂ are both hydrogen or taken together R₁ and R₂ are ═O;
R₃ is chosen from hydrogen, alkyl, substituted alkyl, alkenyl, substituted alkenyl, alkynyl, substituted alkynyl, aryl, substituted aryl, heterocyclyl and substituted heterocyclyl;
R₄ is chosen from hydrogen, halogen, hydroxy, amino, alkoxy, C₁-C₂₀ alkyl and substituted C₁-C₂₀ alkyl;
R₅ and R₆ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₅ and R₆ together form a carbonyl or a vinyl substituent;
each R₂₀ and R₂₁ is independently selected from hydrogen, halogen, aliphatic, substituted aliphatic, aryl or substituted aryl; alternatively, two R₂₀ groups or one R₂₀ and one R₂₁ groups together with the atoms to which they are attached may form a two, three, four, five, six or seven membered ring; and,
R₇ and R₈ are independently selected from hydrogen, halogen, —OR₂₀, —SR₂₀, —NR₂₀R₂₁, —C(O)R₂₀, —C(O)OR₂₀, —C(O)NR₂₀R₂₁, —N(R₂₀)C(O)R₂₁, —CF₃, —CN, —NO₂, —N₃, alkyl, substituted alkyl, alkenyl, substituted alkenyl, aryl, substituted aryl, heterocyclyl and substituted heterocyclyl;
alternatively, R₇ and R₈ together form an —O— or —S— group.

46. The method according to claim 30, wherein said subject is administered an additional pharmaceutical agent selected from cholinesterase inhibitors and memantine, or a combination thereof.

47. The method according to claim 46, wherein said cholinesterase inhibitor is selected from the group consisting of tacrine, donepezil, rivastigmine, galantamine, and pharmaceutically acceptable salts thereof. In one embodiment, the second pharmaceutical agent is selected from acamprosate, amlodipine, argatroban, baclofen, cilostazol, cinacalcet, clopidogrel, dyphylline, fenoldopam, leflunomide, mepacrine, methimazole, phenformin, prilocaine, rifabutin, sulfisoxazole, tadalafil, terbinafine, torasemide, cinnarizine, ciclopirox, eplerenone, carbenoxolone, sulodexide, carbamazine, amobarbital, cefotetan, erythrityl tetranitrate, methyclothiazide, risedronate, enprofylline, oxtriphylline, paramethadione, cefmenoxime, aprindine, etomidate, mitiglinide, benidipine, levosimendan, and zonisamide.
